(19) United States
(12) Patent Application Publication
(10) Pub. No.: US 2018/0318347 A1
(43) Pub. Date: Nov. 8, 2018

(54) METHODS FOR TREATING CANCER

(71) Applicant: Agenus Inc., Lexington, MA (US)

(72) Inventors: Jennifer Buell, Jamaica Plain, MA (US); Andrew Parsa, Chicago-Lincoln Park, IL (US); Orin Bloch, Chicago, IL (US)

(73) Assignee: Agenus Inc., Lexington, MA (US)

(21) Appl. No.: 15/568,608

(22) PCT Filed: Apr. 22, 2016
(86) PCT No.: PCT/US16/29038

§ 371 Date: Oct. 23, 2017

Related U.S. Application Data
(60) Provisional application No. 62/257,211, filed on Nov. 18, 2015.
(60) Provisional application No. 62/160,961, filed on May 13, 2015.
(60) Provisional application No. 62/151,398, filed on Apr. 22, 2015.

(51) Int. Cl. A61K 35/15; A61K 31/495; A61K 39/00; A61K 39/395; A61P 35/00; C07K 16/28; A61K 45/06

(52) U.S. Cl. CPC A61K 35/15; A61K 31/495; A61K 39/0011; A61K 39/395; A61K 2300/00; C07K 16/2818; C07K 16/2827; A61K 45/06; A61K 2039/6043; A61P 35/00

(57) ABSTRACT

Aspects of the disclosure relate to methods of treating a subject who has had a Glioblastoma Multiforme (GBM) tumor surgically removed.

### RELATED APPLICATIONS

This application is a national stage filing under U.S.C. § 371 of PCT International Application PCT/US2016/029038, filed Apr. 22, 2016, which claims priority under 35 U.S.C. § 119(e) to U.S. Provisional Application Ser. No. 62/151,398, entitled “METHODS FOR TREATING GLIOBLASTOMA” filed on Apr. 22, 2015, to U.S. Provisional Application Ser. No. 62/160,961, entitled “METHODS FOR TREATING CANCER” filed on May 13, 2015, and U.S. Provisional Application Ser. No. 62/257,211 entitled “METHODS FOR TREATING CANCER” filed on Nov. 18, 2015 which are herein incorporated by reference in their entireties.

### BACKGROUND

Glioblastoma (GBM) is the most common primary brain malignancy. The current standard of care for GBM at initial diagnosis is surgical resection followed by radiotherapy and temozolomide, as described in Stupp, R. et al., N Engl J Med 2005; 352:987-96 and Stupp, R. et al. LancetOnc 2009. However, there remains a need for improved methods for treating GBM and other cancers.

### SUMMARY

According to some aspects of the disclosure, methods are provided for treating a subject having cancer, e.g., a Glioblastoma Multiforme (GBM) tumor. In some embodiments, methods provided herein involve administering to the subject an autologous heat-shock protein peptide complex that comprises peptides derived from the GBM tumor complexed with heat-shock proteins in combination with surgical removal of the GBM tumor along with radiotherapy and temozolomide, as prescribed by the standard of care. Particular aspects of the disclosure relate to a surprising discovery that this combination treatment (autologous heat-shock protein peptide complex and temozolomide) results in 1) a median overall survival of approximately 23.8 months, which is significantly greater than the median overall survival achieved with the standard of care alone; 2) a median overall survival for patients with low PD-L1 expression on circulating myeloid lineage cells (e.g., circulating CD45+/CD11b+ cells, e.g., myeloid derived suppressor cells, leukocytes, monocytes) of approximately 44.7 months, which is significantly greater than the median overall survival of approximately 18.0 months observed in high PD-L1 expressors; and 3) a post progression survival of approximately 17.5 months for patients with low PD-L1 expression on circulating myeloid lineage cells (e.g., circulating CD45+/CD11b+ cells, e.g., myeloid derived suppressor cells, leukocytes, monocytes), which is significantly greater than the post progression survival of approximately 6-12 months for all subjects regardless of PD-L1 expression status.

In some embodiments, methods provided herein comprise administering to a subject who has had a GBM tumor surgically removed or resected an autologous heat-shock protein peptide complex that comprises peptides derived from the GBM tumor complexed with heat-shock proteins, in which the subject exhibits low PD-L1 expression on circulating myeloid lineage cells (e.g., circulating CD45+/CD11b+ cells, e.g., myeloid derived suppressor cells, leukocytes, monocytes) derived from a sample of the subject's blood taken around the time of surgical removal (e.g., within 24 hours of said surgical removal) of the GBM tumor or taken within 10 days of surgical removal of the GBM tumor; and in some embodiments, the subject survives at least 36 months following surgical removal of the GBM tumor. In some embodiments, 60% or less, 54.5% or less, or 50% or less of the cells (e.g., peripheral monocytes) are PD-L1 positive. In some embodiments, the cells are peripheral monocytes. In certain embodiments, the peripheral monocytes are selected from the group consisting of CD45+ monocytes, CD11b+ monocytes, and CD45+/CD11b+ monocytes. In some embodiments, the subject survives in a range of 36 months to 48 months, 36 months to 60 months, 36 months to 72 months or 36 months to 96 months following surgical removal of the GBM tumor. In some embodiments, the subject at least 33 months, at least 34 months, at least 35 months, at least 36 months, at least 37 months, at least 38 months, at least 39 months, at least 40 months, at least 41 months, at least 42 months, at least 43 months, at least 44 months, at least 44.7 months, at least 45 months, at least 46 months, at least 47 months, at least 48 months, at least 60 months or more following surgical removal of the GBM tumor.

According to some aspects of the disclosure, methods are provided for treating a subject who has had a Glioblastoma Multiforme (GBM) tumor surgically removed or resected, in which the methods involve selecting a subject as a candidate for a treatment that comprises administration of an autologous heat-shock protein peptide complex that comprises GBM tumor peptides complexed with heat-shock proteins, in which the selection is based on a determination that the subject is a member of a population (e.g., a population of predicted responders) having a median overall survival rate of at least 36 months following surgical removal of the GBM tumor in response to the treatment; and, based on the selection, administering to the subject the autologous heat-shock protein peptide complex. In some embodiments, the subject survives in a range of 36 months to 48 months, 36 months to 60 months, 36 months to 72 months or 36 months to 96 months following surgical removal of the GBM tumor. In some embodiments, the subject survives at least 33 months, at least 34 months, at least 35 months, at least 36 months, at least 37 months, at least 38 months, at least 39 months, at least 40 months, at least 41 months, at least 42 months, at least 43 months, at least 44 months, at least 44.7 months, at least 45 months, at least 46 months, at least 47 months, at least 48 months, at least 60 months or more following surgical removal of the GBM tumor.

According to some aspects of the disclosure, methods are provided for treating a subject who has had a Glioblastoma Multiforme (GBM) tumor surgically removed or resected, in which the methods involve selecting a subject as a candidate for a treatment that comprises administration of an autologous heat-shock protein peptide complex that comprises GBM tumor peptides complexed with heat-shock proteins, in which the selection is based on a determination that the subject is a member of a population having a median post-progression survival of at least 6 months in response to the treatment; and, based on the selection, administering to the subject the autologous heat-shock protein peptide complex. In some embodiments, the subject survives, post-progression, in a range of 6 months to 12 months, 6 months to 24 months, 6 months to 72 months, 12 months to 18 months, 12 months to 24 months, 12 months to 72 months, 12 months to 96 months, 24 months to 72 months, or 24 months to 96 months. In some embodiments, the subject survives, post-progression, at least 8 months, at least 10 months, at least 12 months, at least 18 months, at least 24 months, at least 36 months, at least 48 months, or at least 72 months.

According to other aspects of the disclosure, methods are provided for treating a subject having a Glioblastoma Multiforme (GBM) tumor, in which the methods involve administering to the subject an autologous heat-shock protein peptide complex that comprises peptides derived from the GBM tumor complexed with heat-shock proteins, in which a sample of the subject's blood (e.g., a peripheral blood sample) was obtained from the subject, prior to the administering step, in which it was determined from the blood sample that less than a threshold level of circulating myeloid lineage cells (e.g., circulating CD45+/CD11b+ cells, e.g., myeloid derived suppressor cells, leukocytes, monocytes) in peripheral blood of the subject were PD-L1 positive, and in which the subject was selected as a candidate for administration of the autologous heat-shock protein peptide complex based on that determination that the subject has an increased likelihood of overall survival of at least 36 months. In some embodiments, the subject survives at least 36 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 44.7 months, at least 45 months, at least 46 months, at least 47 months, at least 48 months, at least 60 months or more following surgical removal of the GBM tumor. In some embodiments, 60% or less, 54.5% or less, or 50% or less of the cells (e.g., peripheral monocytes) are PD-L1 positive. In some embodiments, the cells are peripheral monocytes. In certain embodiments, the peripheral monocytes are selected from the group consisting of CD45+ monocytes, CD11b+ monocytes, and CD45+/CD11b+ monocytes. In some embodiments, the CD45+/CD11b+ cells are CD14+ monocytes.

In some embodiments, a subject is selected for administration of the autologous heat-shock protein peptide complex based on a further determination that the GBM tumor is MGMT promoter methylation positive.

In some embodiments, a sample of the subject's blood (e.g., a peripheral blood sample) is obtained within 24 hours prior to surgical resection of the GBM tumor. In some embodiments, a sample of the subject's blood (e.g., a peripheral blood sample) was obtained within 24 hours after surgical resection of the GBM tumor.

According to other aspects of the disclosure, methods are disclosed for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, in which, prior to the administering step, it was determined that GBM tumor was MGMT promoter methylation positive, and wherein the subject was selected as a candidate for administration of the autologous heat-shock protein peptide complex based on that determination.

According to some aspects of the disclosure, methods are provided for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, such that the subject survives at least 36 months following surgical removal of the GBM tumor, wherein the subject is selected for the treatment based on detection of low PD-L1 expression on peripheral leukocytes derived from a sample of the subject's blood (e.g., blood taken within 24 hours or within 10 days of said surgical removal of said GBM tumor).

According to some aspects of the disclosure, methods are provided for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, and in which (i) the subject exhibits low PD-L1 expression on CD45+/CD11b+ cells derived from a sample of the subject's blood (e.g., blood taken within 24 hours or within 10 days of said surgical removal of said GBM tumor); and (ii) the subject survives at least 36 months or at least 44.7 months following surgical removal of the GBM tumor. In some embodiments, 60% or less of the CD45+/CD11b+ cells are PD-L1 positive. In some embodiments, 54.5% or less of the CD45+/CD11b+ cells are PD-L1 positive. In some embodiments, 50% or less of the CD45+/CD11b+ cells are PD-L1 positive. In some embodiments, blood is taken within 1 hour of said surgical removal of said GBM tumor. In some embodiments, blood is taken at the time of said surgical removal of said GBM tumor.

In some aspects, methods are provided for treating Glioblastoma Multiforme (GBM) that involve administering an autologous heat-shock protein peptide complex that comprises peptides derived from the GBM tumor complexed with heat-shock proteins to a subject who i) has had a GBM tumor surgically removed and ii) exhibits low PD-L1 expression on CD45+/CD11b+ cells, such that the subject survives at least 36 months or 44.7 months following surgical removal of the GBM tumor. In some embodiments, the CD45+/CD11b+ cells are CD14+. In some embodiments, 60% or less of the CD45+/CD11b+ cells are PD-L1 positive. In some embodiments, 54.5% or less of the CD45+/CD11b+ cells are PD-L1 positive. In some embodiments, 50% or less of the CD45+/CD11b+ cells are PD-L1 positive.

In some aspects, methods are provided for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, such that the subject survives at least 36 months following surgical removal of the GBM tumor, wherein the subject is selected for the treatment based on detection of i) low PD-L1 expression on peripheral leukocytes derived from a sample of the subject's blood (e.g., blood taken within 24 hours or within 10 days of said surgical removal of said GBM tumor), and ii) high MGMT promoter methylation in cells of the GBM tumor. In some embodiments, the subject survives at least 44.7 months following surgical removal of the GBM tumor. In some embodiments, 60% or less, 54.5% or less, or 50% or less of the peripheral leukocytes are PD-L1 positive. In some embodiments, the peripheral leukocytes are selected from the group consisting of CD45+ leukocytes, CD11b+ leukocytes, and CD45+/CD11b+ leukocytes.

In some aspects, methods are provided for treating a subject having a Glioblastoma Multiforme (GBM) tumor, in which the methods involve administering to the subject an autologous heat-shock protein peptide complex that comprises peptides derived from the GBM tumor complexed with heat-shock proteins, and administering to the subject an effective amount of a PD-1 inhibitor and/or PD-L1 inhibitor. In some embodiments, the PD-1 inhibitor is an anti-PD-1 antibody, e.g., lambrolizumab (also known as pembrolizumab). In some embodiments, the PD-L1 inhibitor is an anti-PD-L1 antibody, e.g., durvalumab. Other examples of PD-1 inhibitors and PD-L1 inhibitors are disclosed herein.

In some embodiments, an effective amount of the PD-1 inhibitor or PD-L1 inhibitor is administered prior to the autologous heat-shock protein peptide complex. In some embodiments, an effective amount of the PD-1 inhibitor or PD-L1 inhibitor is administered up to 1 day, 2 days, 5 days, 1 week, 1 month or more prior to administration of the autologous heat-shock protein peptide complex. In some embodiments, the PD1 inhibitor or PD-L1 inhibitor is administered at or around the time of surgery to remove the GBM tumor. In some embodiments, the PD1 inhibitor or PD-L1 inhibitor is administered prior to the time of surgery to remove the GBM tumor. In some embodiments, the PD1 inhibitor or PD-L1 inhibitor is administered after determination of PD-L1 expression on circulating monocytes.

In some embodiments, an effective amount of the PD-1 inhibitor or PD-L1 inhibitor is administered after administration of the autologous heat-shock protein peptide complex. In some embodiments, an effective amount of the PD-1 inhibitor or PD-L1 inhibitor is administered up 1 day, 2 days, 5 days, 1 week, 1 month or more after to the autologous heat-shock protein peptide complex. However, in some embodiments, an effective amount of the PD-1 inhibitor or PD-L1 inhibitor is administered concurrently with administration of the autologous heat-shock protein peptide complex.

In some embodiments, multiple doses of a PD1 inhibitor and/or PD-L1 inhibitor are administered. For example, in some embodiments, multiple doses of a PD1 inhibitor or PD-L1 inhibitor are administered after removal of the GBM tumor. In some embodiments, multiple doses of the PD1 inhibitor or PD-L1 inhibitor are administered after removal of the GBM tumor but before administration of the autologous heat-shock protein peptide complex.

In some embodiments, the PD1 inhibitor and/or PD-L1 inhibitor is administered to a subject in combination with a prescribed standard of care, including, for example, surgical removal of the GBM tumor, radiotherapy and temozolomide. In some embodiments, the subject is also administered the autologous heat-shock protein peptide complex.

In some aspects, methods are provided for treating a subject having a Glioblastoma Multiforme (GBM) tumor, in which the methods involve administering to the subject an autologous heat-shock protein peptide complex that comprises peptides derived from the GBM tumor complexed with heat-shock proteins, wherein it was determined from a sample of the subject's blood (e.g., a peripheral blood sample obtained from the subject) that greater than a threshold level of circulating CD45+/CD11b+ cells in blood (e.g., peripheral blood) of the subject were PD-L1 positive; and administering to the subject an effective amount of a PD-1 inhibitor or PD-L1 inhibitor to the subject. In some embodiment, the blood sample was obtained from the subject within 24 hours or within 10 days of surgically resecting the GBM tumor. In some embodiments, the CD45+/CD11b+ cells are CD14+. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75%. In some embodiments, the threshold level is 50%, 55%, 60%, 65% or 70%. In some embodiments, the threshold level is 60%, 54.5%, or 50%.

In some aspects, methods are provided for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, wherein the subject exhibits high PD-L1 expression on cells derived from a sample of the subject's blood (e.g., blood taken within 24 hours or within 10 days of said surgical removal of said GBM tumor). In some embodiments, the methods further involve administering to the subject an effective amount of a PD-1 inhibitor or PD-L1 inhibitor to the subject. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75%. In some embodiments, the threshold level is 50%, 55%, 60%, 65% or 70%. In some embodiments, the threshold level is 60%, 54.5%, or 50%. In some embodiments, the cells are CD45+ cells, CD11b+ cells, or CD45+/CD11b+ cells. In some embodiments, the cells are CD14+ cells.

In some embodiments, the CD45+/CD11b+ cells are derived from a sample of the subject's blood. In some embodiments, the sample of the subject's blood is taken within 10 days of the surgical removal of the GBM tumor. In some embodiments, the sample of the subject's blood is taken within 1 week of the surgical removal of the GBM tumor. In some embodiments, the sample of the subject's blood is taken within 24 hours of the surgical removal of the GBM tumor. In some embodiments, the sample of the subject's blood is taken within 24 hours prior to the surgical removal of the GBM tumor. In some embodiments, the sample of the subject's blood is taken within 24 hours after the surgical removal of the GBM tumor. In some embodiments, the sample of the subject's blood is taken within 1 hour of the surgical removal of the GBM tumor. In some embodiments, the sample of the subject's blood is taken at the time of the surgical removal of the GBM tumor. In some embodiments, the PD-L1 expression is measured by flow cytometry.

In some embodiments, a PD-1 inhibitor is an anti-PD-1 antagonist antibody or antigen binding fragment thereof. In some embodiments, a PD-L1 inhibitor is an anti-PD-L1 antagonist antibody or antigen binding fragment thereof. In some embodiments, the PD-1 inhibitor is pembrolizumab or nivolumab. In some embodiments, an antibody or antigen binding fragment thereof is administered intravenously.

In some embodiments, a subject is administered radiotherapy directed at the area from which the GBM tumor was resected. In other embodiments, radiotherapy is completed within 5 weeks to seven weeks (e.g., 6 weeks) of the autologous heat-shock protein peptide complex administration. In some embodiments, radiotherapy comprises 60 Gy being administered in 2 Gy fractions. In some embodiments, 2 Gy fractions are administered 4 to 6 days a week for 5 to 7 weeks. In some embodiments, 2 Gy fractions are administered 5 days a week for 6 weeks. In some embodiments, radiotherapy is completed within 2 weeks to 5 weeks before the first autologous heat-shock protein peptide complex administration.

In some embodiments, a subject is further administered oral temozolomide to treat the GBM tumor. In some embodiments, oral temozolomide is administered at a dose of 75 mg per square meter of body-surface area. In some embodiments, oral temozolomide doses are administered daily for up to 49 days.

In other embodiments, prior to resection of the GBM tumor, the subject had a Karnofsky performance status of at least 70.

In some embodiments, the extent of surgical resection of the GBM tumor is in excess of 90%. In some embodiments the extent of surgical resection is as determined by detection of contrast-enhancing tumor, e.g., residual contrast-enhancing tumor, on post-operative MRI within 30 days of surgery, e.g., within 3 days of surgery, or within 28 days of surgery.

In some embodiments, sufficient tissue is obtained from a resected GBM tumor to generate multiple doses of the autologous heat-shock protein peptide complex. In certain embodiments, sufficient tissue is obtained from surgically resected GBM tumor to generate a minimum of four 25 μg doses of the autologous heat-shock protein peptide complex. In some embodiments, a subject to be treated did not have a concurrent malignancy within the past 5 years of the treatment.

In some embodiments, methods provided herein further comprise administering the autologous heat-shock protein peptide complex once a week for the first 4 weeks of administration. In some embodiments, methods provided herein further comprise administering the autologous heat-shock protein peptide complex once every other week after the first 4 weeks of administration. In some embodiments, the autologous heat-shock protein peptide complex and the temozolomide are administered 2 to 4 days apart, 4 to 6 days apart, 1 week apart, 1 to 2 weeks apart, or 2 to 4 weeks apart. In some embodiments, the autologous heat-shock protein peptide complex and temozolomide are administered concurrently. In some embodiments, the autologous heat-shock protein peptide complex and temozolomide are administered cyclically. In some embodiments, the autologous heat-shock protein peptide complex and temozolomide are administered for up to 12 cycles.

In some embodiments, methods provided herein further comprise administering at least one maintenance dose of temozolomide following completion of radiotherapy and administration of autologous heat-shock protein peptide complex. In some embodiments, the at least one maintenance dose of temozolomide is up to 150 mg per square meter of body-surface area. In some embodiments, methods provided herein further comprise administering at least one escalated maintenance dose of temozolomide following completion of radiotherapy and administration of the autologous heat-shock protein peptide complex. In certain embodiments, the at least one escalated maintenance dose of temozolomide is in a range of greater than 150 mg per square meter of body-surface area and 200 mg per square meter of body-surface area. In some embodiments, the at least one maintenance dose of temozolomide is administered two to five weeks following completion of radiotherapy and administration of the autologous heat-shock protein peptide complex. In certain embodiments, methods provided herein further comprise administering at least one maintenance dose of temozolomide following completion of radiotherapy and a fourth administration of the autologous heat-shock protein peptide complex. In some embodiments, methods provided herein further comprise administering a maintenance dose of temozolomide two weeks following completion of radiotherapy and a fourth administration of the autologous heat-shock protein peptide complex. In certain embodiments, the maintenance dose of temozolomide is administered on the same day as a fifth administration of the autologous heat-shock protein peptide complex.

In certain embodiments, the autologous heat-shock protein peptide complex comprises heat shock protein-peptide complexes that comprise one or more of hsp70, hsc70, hsp90, hsp110, gp96, grp170, and calreticulin. In certain embodiments, the autologous heat-shock protein peptide complex comprises heat shock protein-peptide complexes that comprise gp96.

In some embodiments, the autologous heat-shock protein peptide complex comprises is administered by intradermal injection. In some embodiments, the autologous heat-shock protein peptide complex is administered in a total volume of 0.3 to 0.6 mL. In some embodiments, the injection is administered at one site. In some embodiments, the injection is administered at more than one site. In some embodiments, the injection is administered in approximate equal volumes at two or more sites. In some embodiments, at least one site of injection is selected from: an anterior deltoid region and a subclavicular region bilaterally. In some embodiments, areas distal to lymph node basins that have been resected or irradiated or areas just distal to a surgical scar are not injected. In some embodiments, the sites of injections are rotated, so that injections are not repeated at the same site twice in a row.

In some embodiments, the GBM tumor peptides, or peptides derived from the GBM tumor, are peptides isolated directly from a surgically resected GBM tumor of the subject. In some embodiments, the GBM tumor peptides, or peptides derived from the GBM tumor, are synthetic peptides comprising amino acid sequences of neoepitopes present in a surgically resected GBM tumor of the subject.

Aspects of the disclosure provide autologous heat-shock protein peptide complex for use in the treatment of Glioblastoma Multiforme (GBM) in a subject, wherein the subject (a) has had a GBM tumor surgically removed; and (b) exhibits low PD-L1 expression on peripheral monocytes derived from a sample of the subject's blood (e.g., blood taken within 24 hours or within 10 days of said surgical removal of said GBM tumor); and wherein the autologous heat-shock protein peptide complex comprises peptides derived from the GBM tumor surgically removed from the subject complexed with heat-shock proteins. In some embodiments, the peripheral monocytes are CD45+ monocytes. In some embodiments, the peripheral monocytes are CD11b+ monocytes.

In some embodiments, the sample of the subject's blood is taken within 10 days of the surgical removal of the GBM tumor. In some embodiments, the sample of the subject's blood is taken within 1 week of the surgical removal of the GBM tumor. In some embodiments, the sample of the subject's blood is taken within 24 hours of the surgical removal of the GBM tumor. In some embodiments, the peripheral monocytes are selected from the group consisting of CD45+ monocytes, CD11b+ monocytes, and CD45+/CD11b+ monocytes. In some embodiments, 60% or less, 54.5% or less, or 50% or less of the peripheral monocytes are PD-L1 positive. In some embodiments, the tumor removed from the subject comprises GBM tumor cells that are MGMT promoter methylation positive.

Aspects of the disclosure provide autologous heat-shock protein peptide complex for use in the treatment of Glioblastoma Multiforme (GBM) in a subject, wherein the subject has (a) had a GBM tumor surgically removed; and (b) the GBM tumor was MGMT promoter methylation positive; and wherein the autologous heat-shock protein peptide complex comprises peptides derived from the GBM tumor surgically removed from the subject complexed with heat-shock proteins. In some embodiments, the complex comprises at least a 96 kDa heat shock protein. In some embodiments, the subject has also been administered at least one dose of radiotherapy directed to the area from which the GBM tumor was surgically removed. In some embodiments, the complex is administered to the subject in combination with radiotherapy. In some embodiments, the radiotherapy is completed within 5 weeks of the complex being administered to the subject. In some embodiments, the radiotherapy comprises 60 Gy being administered in 2 Gy fractions. In some embodiments, the 2 Gy fractions are administered 4 to 6 days a week for 5 to 7 weeks. In some embodiments, the subject has also been administered at least one dose of oral temozolomide. In some embodiments, the complex is administered to the subject in combination with oral temozolomide. In some embodiments, the oral temozolomide is administered at a dose of 75 mg per square meter of body-surface area. In some embodiments, the oral temozolomide doses are administered daily for up to 49 days. In some embodiments, prior to surgical removal of the GBM tumor the subject had a Karnofsky performance status of at least 70. In some embodiments, the extent of surgical removal of the GBM tumor is in excess of 90%. In some embodiments the extent of surgical resection is as determined by detection of contrast-enhancing tumor, e.g., residual contrast-enhancing tumor, on post-operative MRI within 30 days of surgery, e.g., within 3 days of surgery, or within 28 days of surgery. In some embodiments, sufficient tissue was obtained from the surgically removed GBM tumor to generate multiple doses of the autologous heat-shock protein peptide complex. In some embodiments, sufficient tissue was obtained from the surgically removed GBM tumor to generate a minimum of four 25 μg doses of the autologous heat-shock protein peptide complex.

Aspects of the disclosure provide methods of identifying a therapeutically effective treatment for a subject having Glioblastoma Multiforme (GBM); said subject having had a GBM tumor surgically removed; comprising the steps of: (a) providing a sample of either the subject's blood comprising peripheral monocytes or peripheral monocytes derived from a sample of the subject's blood (e.g., a blood sample taken within 24 hours or within 10 days of surgical removal of the GBM tumor); (b) quantifying PD-L1 expression on the peripheral monocytes; (c) when the PD-L1 expression is low, identifying an autologous heat-shock protein peptide complex as defined herein as the appropriate treatment (involving an autologous heat-shock protein peptide complex administration) for the subject. In some embodiments, the peripheral monocytes are CD45+ monocytes. In some embodiments, the peripheral monocytes are CD11b+ monocytes. In some embodiments, the sample of the subject's blood is taken within 10 days of the surgical removal of the GBM tumor. In some embodiments, the sample of the subject's blood is taken within 1 week of the surgical removal of the GBM tumor. In some embodiments, the sample of the subject's blood is taken within 24 hours of the surgical removal of the GBM tumor. In some embodiments, the peripheral monocytes are selected from the group consisting of CD45+ monocytes, CD11b+ monocytes, and CD45+/CD11b+ monocytes. In some embodiments, 60% or less, 54.5% or less, or 50% or less of the peripheral monocytes are PD-L1 positive. In some embodiments, the PD-L1 expression is measured by flow cytometry.

Aspects of the disclosure provide methods of identifying a therapeutically effective treatment for a subject having Glioblastoma Multiforme (GBM); said subject having had a GBM tumor surgically removed; comprising the steps of: (a) providing a sample of the GBM tumor surgically removed from the subject; (b) identifying the methylation status of MGMT promoter; and (c) when the MGMT promoter is methylation positive, identifying an autologous heat-shock protein peptide complex as defined herein as the appropriate treatment (involving an autologous heat-shock protein peptide complex administration) for the subject. In some embodiments, the methods involve the step of formulating an autologous heat-shock protein peptide complex, comprising preparing for intradermal injection a composition comprising an autologous heat-shock protein peptide complex comprising peptides derived from the GBM tumor surgically removed from the subject complexed with heat-shock proteins. In some embodiments, the methods involve the step of formulating an autologous heat-shock protein peptide complex, comprising complexing at least one peptide having a neoepitope derived from the GBM tumor surgically removed from the subject with at least one recombinant heat-shock protein.

### BRIEF DESCRIPTION OF DRAWINGS

FIGS. 1A-1B show non-limiting examples of progression-free survival and overall survival graphs. Kaplan-Meier estimates of progression-free survival (panel A) and overall survival (panel B) in the intention-to-treat population are shown. Vertical bars indicate time points at which patients were censored and dotted-line curves indicate the 95% confidence interval.

FIGS. 2A-2B show non-limiting examples of progression-free survival and overall survival graphs by frequencies of PD-L1 positive monocytes. Kaplan-Meier estimates of progression-free survival (panel A) and overall survival (panel B) in patients subdivided based upon the frequency of PD-L1-positive monocytes in whole blood (n=32) are shown. The PD-L1 high group had frequencies of PD-L1 positive monocytes greater than the median (54.5% PD-L1+ monocytes) compared to the PD-L1 low group that had frequencies of PD-L1 positive monocytes less than or equal to the median. Vertical bars indicate time points at which patients were censored.

FIGS. 3A-3B show progression-free survival and overall survival by MGMT methylation status. Kaplan-Meier estimates of progression-free survival (panel A) and overall survival (panel B) in patients with available MGMT methylation status (n=42) are shown. Vertical bars indicate time points at which patients were censored.

FIG. 4 is a graph depicting overall survival by PD-L1 expression level and MGMT methylation status.

FIG. 5 is a graph depicting overall survival by monocyte PD-L1 levels.

FIG. 6 is a graph depicting overall survival of treated patients by MGMT status.

### DETAILED DESCRIPTION OF DISCLOSURE

Glioblastoma is the most common primary brain malignancy. The current standard of care for Glioblastoma Multiforme (GBM) at initial diagnosis is surgical resection followed by radiotherapy and temozolomide. Recent studies investigating the addition of bevacizumab to radiation and temozolomide have been disappointing, with no improvement in overall survival compared to placebo. Aspects of the disclosure relate to the recognition that while surgical removal of recurrent GBM tumors is common practice, the resulting benefit is limited when it comes to extending survival. Further aspects of the disclosure relate to the recognition that existing therapeutic options are also limited post-surgery for GBM tumors. Accordingly, in some aspects of the disclosure, resected tumors are utilized to create a personalized, highly multivalent vaccine. In some embodiments, methods are provided for treating subjects (patients) who have surgically resectable recurrent GBM. In some embodiments, methods are provided for treating subjects (e.g., humans, patients) who have residual disease after resection of the GBM tumors. In some embodiments, these subjects are treated with Heat-Shock Protein Peptide Complex-96 (HSPPC-96) in combination with temozolomide and radiotherapy.

In some embodiments, methods provided herein involve administering to a subject an autologous heat-shock protein peptide complex that comprises peptides derived from a GBM tumor complexed with heat-shock proteins in combination with surgical removal of the GBM tumor, radiotherapy and temozolomide, as prescribed by the standard of care. An autologous heat-shock protein peptide complex may comprise peptides isolated directly from a surgically resected GBM tumor of the subject in a complex with heat-shock proteins. Alternatively, in some embodiments, an autologous heat-shock protein peptide complex may comprise synthetic peptides comprising amino acid sequences of neoepitopes present in a surgically resected GBM tumor of the subject. Such neoepitopes may be identified bioinformatically by sequencing the whole genome, the exome (e.g., the coding region of the genome) and/or RNA of a GBM tumor and the exome of healthy tissue from the same patient to identify mutations in the tumor that are predicted to result in expression of T cell neoepitopes by that tumor. Such synthetic peptides may be complexed in vitro to recombinant heat shock protein (e.g., Hsc70 or Hsp70) for administration to a GBM patient.

In some embodiments, these subjects are treated with Heat-Shock Protein Peptide Complex-96 (HSPPC-96) in combination with temozolomide and radiotherapy.

Heat-shock proteins (HSP), which function as intracellular chaperones, can be used to deliver tumor antigens for immune stimulation. Tumor proteins bound to the gp96 HSP can be internalized by antigen presenting cells through the CD91 receptor, resulting in cleavage and cross-presentation of antigenic peptides on MHC class I and II. By purifying HSP complexes from a patient's tumor, an autologous, polyvalent vaccine can be developed and administered for treatment. The safety and efficacy of a heat shock protein peptide complex-96 vaccine (HSPPC-96, Prophage) has been studied in phase 1 and phase 2 single-arm trials for the treatment of recurrent GBM. These studies demonstrated robust peripheral immune stimulation in response to vaccination and modest improvements in survival compared to historical standards. Aspects of the present disclosure relate to evaluation of HSPPC-96 vaccination in combination with standard radiation and chemotherapy for the treatment of newly diagnosed GBM. In some embodiments, PD-L1 expression in circulating leukocytes (e.g., monocytes) on clinical outcomes has been identified as a useful marker for selecting patients for treatment with HSPPC-96.

Aspects of the disclosure relate to methods for treating GBM. However, in some embodiments, methods provided herein that involve treatment with autologous heat-shock protein peptide complex (e.g., HSPPC-96) can be applied to other cancers. For example, such treatment methods may be applied to treating temozolomide responsive cancers such as anaplastic astrocytoma, metastatic melanoma, and oligodendroglioma. In some embodiments, methods provided herein involve administering to a subject an autologous heat-shock protein peptide complex that comprises peptides derived from a tumor of an astrocytoma, metastatic melanoma, or oligodendroglioma complexed with heat-shock proteins in combination with one or more of: surgical removal of the tumor (e.g., at least 90% resection of the tumor), radiotherapy and temozolomide.

In some embodiments, treatment methods disclosed herein may be applied to treating cancer such as non-small cell lung carcinoma, melanoma, inflammatory breast cancer or ovarian cancer for which, in view of data provided herein, PD-L1 levels are informative for treatment selection. For example, in some embodiments, subjects having non-small cell lung carcinoma, melanoma, inflammatory breast cancer or ovarian cancer may be selected for treatment with an autologous heat-shock protein peptide complex on the basis of low PD-L1 levels on CD11b⁺, CD45⁺ cells (e.g., as assessed at the time of surgical removal of a tumor of the cancer).

In some embodiments, treatment methods disclosed herein may be applied to treating cancer such as colorectal cancer for which, in view of data provided herein, MGMT promoter methylation levels are informative for treatment selection. For example, in some embodiments, subjects having colorectal cancer may be selected for treatment with an autologous heat-shock protein peptide complex on the basis of high MGMT promoter methylation levels in a tumor of the cancer.

### Heat-Shock Protein Peptide Complex-96 (HSPPC-96)

In some embodiments, multivalent vaccines provided herein for the treatment of GBM comprise autologous tumor-derived heat-shock protein peptide complex-96 (HSPPC-96). HSPPC-96 is an autologous tumor derived vaccine comprising the 96-kDa heat shock protein gp96 in complex with autologous tumor derived peptides. HSPs are highly conserved, abundant, nonpolymorphic stress proteins physiologically expressed in every cell. They have the function of chaperoning proteins and peptides intracellularly within different compartments; hence they bind to the intrinsic antigenic repertoire of a cell, which can be defined as the antigenic fingerprint. HSPPC-96 preparations activate T cell responses to the chaperoned peptides and to the tumors from which the complexes were derived in animal models and in human cancer patients. The specific immunogenicity and antitumor activity of this complex has been demonstrated in preclinical models, both in prophylaxis and therapy settings as well as in clinical trials. To date, results from single arm Phase 1 and 2 clinical trials in recurrent glioblastoma have shown the vaccine to be well tolerated and to be immunogenic inducing both activation of the innate as well as adaptive immune response. Relative to historical controls the vaccine also appears to provide clinical benefit as measured by overall survival.

In some embodiments, HSPPC-96 for clinical use comprises the 96-kDa heat shock protein gp96 in complex with autologous tumor-derived peptides. In some embodiments, HSPPC-96 is supplied in a single-use vial as a clear, colorless solution. In some embodiments, it is formulated in a 9% sucrose-potassium phosphate for intradermal (ID) injection. In some embodiments, each vial contains 25 μg of HSPPC-96 in a solution of 9% sucrose-potassium phosphate for intradermal (ID) injection. In some embodiments, the total volume of each vial of HSPPC-96 is 0.47 mL. In some embodiments, the total volume that is administered is 0.4 mL. In some embodiments, HSPPC-96 is administered at dose in a range of 1 μg to 25 μg. In cases where recombinant HSP is complexed with synthetic peptides, e.g., synthetic peptides comprising neoepitopes, a dose of such complexes may comprise recombinant HSP in an amount of up to 10μg, up to 20 μg, up to 30 μg, up to 40 μg, up to 50 μg, up to 60 μg, up to 70 μg, up to 80 μg, up to 90 μg, up to 100 μg, up to 110 μg, up to 120 μg, up to 130 μg, up to 140 μg, up to 150 μg, up to 160 μg, up to 170 μg, up to 180 μg, up to 190 μg, up to 200 μg, up to 210 μg, up to 220 μg, up to 230 μg, up to 240 μg, or up to 250 μg. In some embodiments, recombinant HSP is present in a complex in an amount in a range of 1 μg to 250 μg, e.g., 1 μg, 10 μg, 25 μg, 50 μg, 100 μg. In some embodiments, the ratio of synthetic peptide to recombinant HSP (total peptide:protein) is 1:1, 10:1, 20:1, 50:1, 100:1, 1:10, 1:20, 1:50, or 1:100. In some embodiments, the number of different synthetic peptides in a complex is in a range of 1 to 25, 1 to 50, 1 to 100 or 1 to 200 peptides. In some embodiments, the number of different synthetic peptides in a complex is selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100. In some embodiments, for any given total peptide:protein molar ratio, the molar ratio of any one peptide would be a fraction of that amount. For instance, if a recombinant heat shock protein (e.g., Hsc70) is complexed with n peptides at a 1:1 ratio of total peptide:protein, such that any one peptide is present at a molar ratio of 1/n:1.

In some embodiments, each vaccine vial is labeled with the batch number, patient number, patient initials, and patient date of birth (DOB). In some embodiments, following production of vaccine, vials are shipped to a clinical site on dry ice and are stored at −80° C.±20° C. until administration to the patient. In some embodiments, if, at the time of injection, a vial is still frozen, it will be thawed.

In some embodiments, the total volume of HSPPC-96 within a vial provided to a clinical site is 0.47 mL (this volume includes a 0.07 mL overage). In some embodiments, the total volume administered is 0.4 mL (0.07 mL overage). In some embodiments, the contents may be drawn up into a 1-mL hubless (or with small hub) tuberculin or insulin syringe without bubbles and promptly injected intradermally using an appropriate intradermal needle. In some embodiments, the injection may be given into 1 site or into 2 adjacent sites (0.2 mL each) a few centimeters apart. Syringes with slip-tip detachable needles or luer hubs that hold back greater than 0.1 mL should not be utilized.

In some embodiments, the appropriate sites for vaccination include the anterior deltoid regions, subclavicular region bilaterally, and medial inguinal regions of the upper thighs. In some embodiments, the HSPPC-96 is not administered to areas distal to lymph node basins that have been resected or in areas just distal to a surgical scar. In some embodiments, the injection sites are changed or rotated among multiple injections so injections are not repeated at the same site at 2 consecutive administrations and all potential sites are used for the patient before repeating injections at a previously used injection site.

### Radiotherapy and Temozolomide

Aspects of the disclosure relate to the use of radiotherapy in combination with autologous heat-shock protein peptide complexes (e.g., HSPPC-96) for the treatment of GBM. In some embodiments, radiotherapy comprises fractionated focal irradiation. In some embodiments, fractionated focal irradiation is administered at a dose of 2 Gy per fraction. In some embodiments, the fractions are giving once daily. In some embodiments, the fractions are given five days per week. In some embodiments, the fractions are given over a period of six weeks. In some embodiments, radiotherapy is delivered to a gross tumor volume (following resection) with a 2-to-3 cm margin for the clinical target volume. In some embodiments, radiotherapy is planned with dedicated computed tomography (CT) and three-dimensional planning system. In some embodiments, conformal radiotherapy is delivered with linear accelerators with nominal energy of 6 MV or more.

Aspects of the disclosure relate to the use of temozolomide in combination with autologous heat-shock protein peptide complexes (e.g., HSPPC-96) for the treatment of GBM. Temozolomide is an oral chemotherapy drug. Temozolomide (also referred to as TMZ) is an imidazotetrazine derivative of the alkylating agent dacarbazine. Temozolomide exhibits antineoplastic activity by interfering with DNA replication. Temozolomide has demonstrated activity against recurrent glioma among other solid tumors. In particular, temozolomide is indicated for the treatment of high-grade malignant gliomas. In some embodiments, temozolomide has high bioavailability (e.g., greater than 95% bioavailability) when taken orally In some embodiments, because of its small size and lipophilic properties, temozolomide is able to cross the blood-brain barrier. In some embodiments, concentrations in the central nervous system are approximately 30% of plasma concentrations following oral administration. Temozolomide undergoes rapid chemical conversion in the systemic circulation at physiological pH to the active compound, 5-(3-methyltriazen-1-yl)imidazole-4-carboxamide (MTIC). In some embodiments, once temozolomide has entered the central nervous system, it can be spontaneously converted to an active metabolite. In some embodiments, temozolomide is provided in 5 mg, 20 mg, 100 mg, 140 mg, 180 mg or 250 mg capsules for oral administration.

In some embodiments, temozolomide is given at a dose of 75 mg/m² daily during radiotherapy. In some embodiments, following completion of standard radiation treatment (e.g., 60 Gry) and weekly vaccine administration, maintenance temozolomide is initiated about 2 weeks or 2 to 4 weeks post-vaccine administration. In some embodiments, a starting dose of temozolomide of 150 mg/m² daily for the first cycle of maintenance temozolomide is administered with a single dose escalation to 200 mg/m² daily in subsequent cycles if no treatment-related adverse events (e.g., grade 2 events or greater) are noted. In some embodiments, if maintenance temozolomide is discontinued due to toxicity, patients may continue to be treated with vaccine per protocol.

In some embodiments, if a subject has an absolute neutrophil count of less than 1000 cells/μl or platelet levels less than 50,000 platelets/4 then maintenance temozolomide is ceased until absolute neutrophil counts reach 1500 or more cells/μl or platelet levels reach 100,000 or more platelets/μl. In such embodiments, subsequent temozolomide doses are reduced by 50 mg/m².

In some embodiments, if a subject has an absolute neutrophil count in a range of 1000 cells/μl to 1500 cells/μl or platelets in a range of 50,000 platelets/μl to 100,000 platelets/4 then maintenance temozolomide is ceased until absolute neutrophil counts reach 1500 or more cells/μl or platelet levels reach 100,000 or more platelets/μl. In such embodiments, subsequent temozolomide doses are at the initial maintenance dose.

In some embodiments, if a subject receiving maintenance doses of temozolomide has an absolute neutrophil count of greater than 1500 cells/μl or platelet levels greater than 100,000 platelets/4 the maintenance dose is maintained at, or increased to, 200 mg/m² daily.

In some embodiments, radiotherapy and temozolomide are administered according to methods set forth in Stupp, R. et al., N Engl J Med 2005; 352:987-96, the contents of which relating to such administrations are incorporated herein by reference in their entirety.

In some embodiments, methods provided herein involve confirming disease stability clinically and radiographically following radiation treatment and temozolomide chemotherapy, For example, disease stability may be confirmed by determining an absence or lack of growth of a GBM tumor, e.g., at a site from which a GBM tumor was resected.

In some embodiments, administration of an autologous heat-shock protein peptide complex is performed about 1 week to about 6 weeks or about 2 weeks to about 5 weeks (e.g., 2 week to 5 weeks±2 days) following completion of radiotherapy. In some embodiments, a subject is administered about four weekly injections of an autologous heat-shock protein peptide complex (e.g., HSPPC-96). In some embodiments, one or more further injections of the autologous heat-shock protein peptide complex (e.g., HSPPC-96) are administered. In some embodiments, the one or more further injections of the autologous heat-shock protein peptide complex (e.g., HSPPC-96) is administered about 1 week to about 3 weeks (e.g., 2 weeks (±4 days)) following a fourth injection (e.g., of a set of weekly injections) of the autologous heat-shock protein peptide complex. In some embodiments, a fifth injection of the autologous heat-shock protein peptide complex is administered on the same day as a maintenance temozolomide administration. In some embodiments, monthly injections of the autologous heat-shock protein peptide complex are administered following the fifth injection of the autologous heat-shock protein peptide complex. In some embodiments, monthly injections of the autologous heat-shock protein peptide complex begin 3 weeks (+/−7 days) following the fifth injection, and continue until depletion of vaccine or progression. In some embodiments, if a subject completes a treatment with the autologous heat-shock protein peptide complex that results in depletion of the autologous heat-shock protein peptide complex following more than five injections, temozolomide administration and tumor evaluation procedures will continue for at least 12-24 months or for up to 12-24 months from surgery or until disease progression.

### PD-L1

Aspects of the disclosure relate to the use of programmed cell death ligand 1 as a marker for identifying patients who are strong responders to treatment of GBM tumors with autologous heat-shock protein peptide complexes (e.g., HSPPC-96).

Programmed cell death ligand 1 (PD-L1), also known as cluster of differentiation (CD274) or B7 homolog 1 (B7-H1) is a 40 kDa type 1 transmembrane protein that in humans is encoded by the CD274 gene. PD-L1 is expressed on antigen presenting cells including CD14+ monocytes and is a negative regulator of T-cell function. PD-L1 has also been shown to be expressed on the surface of a variety of cancer cells. PD-L1 interacts with its receptor, PD1, expressed on T cells. Normally the immune system reacts to foreign antigens where there is some accumulation in the lymph nodes or spleen which triggers a proliferation of antigen-specific CD4+ and CD8+ T cells. The formation of PD-1 receptor/PD-L1 ligand complex transmits an inhibitory signal which reduces the proliferation of these CD4+ and CD8+ T cells in the lymph nodes and supplementary to that PD-1 is also able to control the accumulation of antigen specific T cells in the lymph nodes through apoptosis which is further mediated by a down regulation of the gene Bcl-2. In some embodiments, GBM tumors express cell-surface PD-L1. In some embodiments, GBM tumors induce PD-L1 expression in circulating leukocytes (e.g., peripheral blood monocytes), which may promote significant systemic immunosuppression and resistance to vaccination.

In some embodiments, the level of PD-L1 in cells of a subject is used as a biomarker for selecting a subject likely to respond to a vaccination (e.g., HSPPC-96 vaccination) for the treatment of a GBM tumor, e.g., in combination with surgical resection, radiotherapy and temozolomide treatment as disclosed herein. In some embodiments, the level of PD-L1 is evaluated in CD11b⁺, CD45⁺ cells. CD11b⁺, CD45⁺ cells may be isolated for analysis of PD-L1 levels using any appropriate cell isolation method. In some embodiments, CD11b⁺, CD45⁺ cells may be isolated as described in Bloch et al. Clin Cancer Res; 19(12); 3165-75. Alternatively, in other embodiments, CD11b+, CD45+ cells may be isolated using double gradient centrifugation, as described, e.g., in Wahl L M and Smith P D. Isolation of Monocyte/Macrophage Populations. In Current Protocols in Immunology. Indianapolis: John Wiley and Sons Inc. 2:7.6A1-7.6A10 and Menck, K., Behme, D., Pantke, M., Reiling, N., Binder, C., Pukrop, T., et al. Isolation of Human Monocytes by Double Gradient Centrifugation and Their Differentiation to Macrophages in Teflon-coated Cell Culture Bags. J. Vis. Exp. (91), e51554, doi:10.3791/51554 (2014). In some embodiments, CD11b+, CD45+ cells may be isolated using flow activated cells sorting (FACS), as described by Wahl L M, et al., 2014, cited above, and Basu S., Campbell H. M., Dittel B. N., Ray A. (2010). Purification of Specific Cell Population by Fluorescence Activated Cell Sorting (FACS). JoVE. 41. In some embodiments, CD11b+, CD45+ cells may be isolated using counterflow centrifugal elutriation (CCE), as described in Wahl L M, et al., 2014, cited above. Double gradient centrifugation and CCE separate cells based upon size. In contrast, FACS involves separating cells based upon the expression of specific surface antigens. All three methods are capable of substantially enriching the cell population of interest. The pertinent contents of the foregoing references are incorporated herein by reference in their entireties.

In some embodiments, the level of PD-L1 in leukocytes is evaluated as a biomarker. In some embodiments, the leukocytes are circulating monocytes (e.g., CD11b⁺, CD45⁺ monocytes of peripheral blood) or tumor infiltrating leukocytes. In other embodiments, the level of PD-L1 expression expressed by cells of a GBM tumor is used as a biomarker for selecting a subject likely to respond to vaccination with HSPPC-96. Accordingly, in some embodiments, the level of PD-L1 in cells of a subject may be used for determining whether a subject is a candidate for a vaccination. In some embodiments, PD-L1 levels are assessed using flow cytometry, immunohistochemistry, an enzyme linked immunosorbent assay (ELISA) or other appropriate immunoassay.

In some embodiments, CD45+, CD11b+ cells are of myeloid lineage, which is a lineage comprising a mixed population of cells that include monocytes, dendritic cells, neutrophils, myeloid derived suppressor cells, etc. In some embodiments, CD45+, CD11b+ cells are circulating CD45+/CD11b+ cells. In some embodiments, CD45+, CD11b+ cells are CD45+, CD11b+ myeloid derived suppressor cells, leukocytes, or monocytes.

In some embodiments, lineage markers may be used to define immune cell populations. In some embodiments, to assess monocytes additional markers may be used, or a pre-enrichment step may be used (for example, the CD14 bead enrichment).

In some embodiments, blood (e.g., peripheral blood, e.g., 30 milliliters of peripheral blood) is obtained for PD-L1 analysis from subjects undergoing surgical resection. In some embodiments, peripheral blood leukocytes are extracted from whole blood by centrifugation, for example, using a Ficoll gradient. In some embodiments, extracted leukocytes are stained with one or more of the following fluorescence-conjugated antibodies to identify immunosuppressive monocytes/macrophages within the total leukocyte population: CD45 FITC, CD11b PE-Cy7, PD-L1 PE (eBioscience, San Diego, Calif.). In some embodiments, cell counts and relative fluorescence are measured by flow cytometry and image analysis (e.g., using a FACSCalibur cytometer (BD Biosciences, San Jose, Calif.) and FlowJo Software (TreeStar, Ashland, Oreg.)). In some embodiments, a subject is categorized as a high PD-L1 expressor if the percentage of PD-L1 positive cells (e.g., circulating monocytes) of the subject is greater than a threshold level. In some embodiments, a subject is categorized as a low PD-L1 expressor if the percentage of PD-L1 positive cells (e.g., circulating monocytes) of the subject is less than or equal to a threshold level. In some embodiments, the threshold level is the median percentage of PD-L1 positive cells (e.g., circulating monocytes) in subjects of an appropriate population of subjects having GBM tumor. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in CD11b⁺, CD45⁺ cells. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in CD11b⁺, CD45⁺ monocytes. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in CD11b⁺, CD45⁺ monocytes of peripheral blood. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in monocytes of peripheral blood. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in CD11b⁺, CD45⁺ tumor infiltrating leukocytes. In some embodiments, the threshold level is 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 54.5%, 55%, 56%, 57%, 58%, 59%, 60%, 61%, 62%, 63%, 64%, 65%, 66%, 67%, 68%, 69%, 70%, 71%, 72%, 73%, 74%, or 75% PD-L1 positive cells in tumor infiltrating lymphocytes. In some embodiments, a threshold level of PD-L1 positive cells in a particular population of cells is about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 54.5%, about 55%, about 56%, about 57%, about 58%, about 59%, about 60%, about 61%, about 62%, about 63%, about 64%, about 65%, about 66%, about 67%, about 68%, 69%, about 70%, about 71%, about 72%, about 73%, about 74%, or about 75%. In this context, the term “about” means±10% of the recited value.

In some embodiments, a subject is defined as a low PD-L1 expressor if less than a threshold level (e.g., listed above) of circulating CD45+/CD11b+ cells from said patient are PD-L1 positive in an assay comprising the following steps:

(a) removing peripheral blood from said subject at the time of surgery;

(b) extracting peripheral blood leukocytes (PBLs) from said peripheral blood;

(c) staining PBLs using an anti-PD-L1 antibody or an isotype control; and an anti-CD45 antibody and an anti-CD11b antibody in a flow cytometry analysis;

(d) gating on CD45+ CD11b+ cells; and

(e) within the CD45+ CD11b+ cells, determining the percentage of PD-L1 positive cells relative to isotype control.

In some embodiments, a subject is defined as a high PD-L1 expressor if more than a threshold level (e.g., listed above) of circulating CD45+/CD11b+ cells from said patient are PD-L1 positive in an assay comprising the following steps:

(a) removing peripheral blood from said subject at the time of surgery;

(b) extracting peripheral blood leukocytes (PBLs) from said peripheral blood;

(c) staining PBLs using an anti-PD-L1 antibody or an isotype control; and an anti-CD45 antibody and an anti-CD11b antibody in a flow cytometry analysis;

(d) gating on CD45+ CD11b+ cells; and

(e) within the CD45+ CD11b+ cells, determining the percentage of PD-L1 positive cells relative to isotype control. In some embodiments, a quantitative PD-L1 threshold is established based on mean fluorescence intensity (e.g., delta MFI—based on isotype control) using calibrated reagents.

In some embodiments, a quantitative PD-L1 threshold is established based on mean receptor quantitation using quantitative flow cytometry (e.g. Dako (http://www.biotech.uiuc.edu/flowcytometry/surface-antigen-quantification), Quantibrite calibration beads (http://www.bdbiosciences.com/ptProduct.jsp?ccn=340495), Bangs laboratory (http://www.bangslabs.com/products/flow-cytometry). In some embodiments, a quantitative PD-L1 threshold is established based on radiolabeled methods for receptor quantitation.

In some embodiments, subjects treated herein exhibit low PD-L1 expression on peripheral monocytes derived from a sample of the subject's blood taken around the time of surgical removal of a GBM tumor (e.g., within 24 hours of surgical removal, e.g., during preoperative or post-operative blood sampling, or at the time of surgery). In some embodiments, a peripheral blood sample is obtained within 30 days, within 4 weeks, within 2 weeks, within 10 days, within 1 week, within 24 hours, within 18 hours, within 12 hours, within 11 hours, within 10 hours, within 9 hours, within 8 hours, within 7 hours, within 6 hours, within 5 hours, within 4 hours, within 3 hours, within 2 hours, within 1 hour, within 30 minutes, within 15 minutes, or within 5 minutes prior to surgical resection of the GBM tumor. In some embodiments, a peripheral blood sample is obtained within 30 days, within 4 weeks, within 2 weeks, within 10 days, within 1 week, within 24 hours, within 18 hours, within 12 hours, within 6 hours, within 3 hours, within 2 hours or within 1 hour after surgical resection of the GBM tumor.

In some embodiments, a peripheral blood sample is obtained at the time of surgical removal. In some embodiments, PD-L1 levels are assessed on isolated circulating monocytes by, e.g., FACS analysis using anti-PD-L1 antibody. In some embodiments, PD-L1 levels are assess during an initial diagnostic assessment of GBM. In some embodiments, selection of treatment for a GBM is based on a baseline expression of PD-L1 levels, e.g., at the time of surgery. Alternatively, in some embodiments, PD-L1 levels are monitored periodically following surgery. In some embodiments, treatment with an autologous heat shock protein complex is indicated when a subject who initially presents with high PD-L1 levels, exhibits low PD-L1 levels subsequent to surgery (e.g., up to 30 days to 60 days following surgery).

In other embodiments, a PD-L1 inhibitor (e.g., an anti-PD-L1 antagonist antibody) and/or PD-1 inhibitor (e.g., an anti-PD-1 antagonist antibody) is administered with an autologous heat-shock protein peptide complex to a subject. In some embodiments, the subject exhibits high PD-L1 levels, e.g., at surgery. In some embodiments, the subject exhibits low PD-L1 levels, e.g., at surgery. In some embodiments, levels of PD-L1 in the subject are not determined or known prior to administration of the PD-L1 inhibitor (e.g., anti-PD-L1 antagonist antibody) and/or PD-1 inhibitor (e.g., anti-PD-1 antagonist antibody).

In some embodiments, a PD-L1 inhibitor used in methods disclosed herein is an anti-PD-L1 antibody or antibody fragment. In some embodiments, an anti-PD-L1 antibody or antibody fragment is administered to a subject as described herein. In some embodiments, an anti-PD-L1 antibody or antibody fragment is administered to a subject as described herein. In some embodiments, the anti-PD-L1 antibody is atezolizumab developed by Genentech. In some embodiments, the anti-PD-L1 antibody is durvalumab developed by AstraZeneca, Celgene and Medimmune. In some embodiments, the anti-PD-L1 antibody is avelumab, also known as MSB0010718C, developed by Merck Serono and Pfizer. In some embodiments, the anti-PD-L1 antibody is MDX-1105 developed by Bristol-Myers Squibb. In some embodiments, the anti-PD-L1 antibody is AMP-224 developed by Amplimmune and GSK.

Non-limiting examples of anti-PD-L1 antibodies that may be used in treatment methods disclosed herein are disclosed in the following patent and applications, which are incorporated herein by reference in their entireties for all purposes: U.S. Pat. No. 7,943,743; U.S. Pat. No. 8,168,179; U.S. Pat. No. 8,217,149; U.S. Pat. No. 8,552,154; U.S. Pat. No. 8,779,108; U.S. Pat. No. 8,981,063; U.S. Pat. No. 9,175,082; U.S. Publication No. US 2010/0203056 A1; U.S. Publication No. US 2003/0232323 A1; U.S. Publication No. US 2013/0323249 A1; U.S. Publication No. US 2014/0341917 A1; U.S. Publication No. US 2014/0044738 A1; U.S. Publication No. US 2015/0203580 A1; U.S. Publication No. US 2015/0225483 A1; U.S. Publication No. US 2015/0346208 A1; U.S. Publication No. US 2015/0355184 A1; and PCT Publication No. WO 2014/100079 A1; PCT Publication No. WO 2014/022758 A1; PCT Publication No. WO 2014/055897 A2; PCT Publication No. WO 2015/061668 A1; PCT Publication No. WO 2015/109124 A1; PCT Publication No. WO 2015/195163 A1; PCT Publication No. WO 2016/000619 A1; and PCT Publication No. WO 2016/030350 A1.

In some embodiments, a PD-1 inhibitor used in methods disclosed herein is an anti-PD-1 antibody or antibody fragment. In some embodiments, an anti-PD-1 antibody or antibody fragment is administered to a subject as described herein. In some embodiments, the anti-PD-1 antibody is Nivolumab, also known as BMS-936558 or MDX1106, developed by Bristol-Myers Squibb. In some embodiments, the anti-PD-1 antibody is Pembrolizumab, also known as Lambrolizumab or MK-3475, developed by Merck & Co. In some embodiments, the anti-PD-1 antibody is Pidilizumab, also known as CT-011, developed by CureTech. In some embodiments, the anti-PD-1 antibody is MEDI0680, also known as AMP-514, developed by Medimmune. In some embodiments, the anti-PD-1 antibody is PDR001 developed by Novartis Pharmaceuticals. In some embodiments, the anti-PD-1 antibody is REGN2810 developed by Regeneron Pharmaceuticals. In some embodiments, the anti-PD-1 antibody is PF-06801591 developed by Pfizer. In some embodiments, the anti-PD-1 antibody is BGB-A317 developed by BeiGene. In some embodiments, the anti-PD-1 antibody is TSR-042 developed by AnaptysBio and Tesaro. In some embodiments, the anti-PD-1 antibody is SHR-1210 developed by Hengrui.

Further non-limiting examples of anti-PD-1 antibodies that may be used in treatment methods disclosed herein are disclosed in the following patents and patent applications, which are incorporated herein by reference in their entireties for all purposes: U.S. Pat. No. 6,808,710; U.S. Pat. No. 7,332,582; U.S. Pat. No. 7,488,802; U.S. Pat. No. 8,008,449; U.S. Pat. No. 8,114,845; U.S. Pat. No. 8,168,757; U.S. Pat. No. 8,354,509; U.S. Pat. No. 8,686,119; U.S. Pat. No. 8,735,553; U.S. Pat. No. 8,747,847; U.S. Pat. No. 8,779,105; U.S. Pat. No. 8,927,697; U.S. Pat. No. 8,993,731; U.S. Pat. No. 9,102,727; U.S. Pat. No. 9,205,148; U.S. Publication No. US 2013/0202623 A1; U.S. Publication No. US 2013/0291136 A1; U.S. Publication No. US 2014/0044738 A1; U.S. Publication No. US 2014/0356363 A1; U.S. Publication No. US 2016/0075783 A1; and PCT Publication No. WO 2013/033091 A1; PCT Publication No. WO 2015/036394 A1; PCT Publication No. WO 2014/179664 A2; PCT Publication No. WO 2014/209804 A1; PCT Publication No. WO 2014/206107 A1; PCT Publication No. WO 2015/058573 A1; PCT Publication No. WO 2015/085847 A1; PCT Publication No. WO 2015/200119 A1; PCT Publication No. WO 2016/015685 A1; and PCT Publication No. WO 2016/020856 A1.

In some embodiments, the foregoing antibodies are administered by any appropriate route, including, for example, intravenously, subcutaneously, intratumorally, or delivered to a tumor draining lymph node.

### Surgical Resection

Generally subjects undergo standard surgical resection of intracranial GBM tumor prior to treatment. In some embodiments, a surgeon or surgical pathologist dissects the specimen in a sterile fashion. In some embodiments, a pathologist or surgeon assesses the viability of the sample and confirm histology as GBM. In such embodiments, tissue is sent for vaccine if it is histologically confirmed as GBM, necrotic, or contains cystic degeneration. Sections of viable tissues are retained until shipment for vaccine production.

In some embodiments, imaging is performed after surgical resection to evaluate the percentage of tumor resected. Collection of tissue for biomarker analyses may also be performed as disclosed herein.

### MGMT Promoter Methylation

In some aspects of the disclosure MGMT promoter methylation status in GBM tumors provides a biomarker useful for selecting subject for treatment with an autologous heat-shock protein peptide complex. In some embodiments, methods are provided herein for treating a subject who has had a Glioblastoma Multiforme (GBM) tumor surgically removed, in which the methods involve administering to the subject an autologous heat-shock protein peptide complex that comprises peptides derived from the GBM tumor complexed with heat-shock proteins, in which, prior to the administering step, was MGMT promoter methylation status was determined. In some embodiments, the subject was selected as a candidate for administration of the autologous heat-shock protein peptide complex based on the determination of MGMT promoter methylation status indicating an increased likelihood of overall survival. In some embodiments, a determination that MGMT promoter methylation is positive indicates an increased likelihood of overall survival of at least approximately 36, at least approximately 40, at least approximately 44, at least approximately 44.7, at least approximately 48, or at least approximately 52 months following surgical resection of the GBM tumor. In some embodiments, a determination that MGMT promoter methylation is negative indicates an increased likelihood of overall survival of at least approximately 14 months, at least approximately 18, or at least approximately 22 months following surgical resection of the GBM tumor.

### Subjects to be Treated

The term “subject,” as used herein, generally refers to a mammal. Typically the subject is a human. However, the term embraces other species, e.g., pigs, mice, rats, dogs, cats, or other primates. In certain embodiments, the subject is an experimental subject such as a mouse or rat. The subject may be a male or female. The subject may be an infant, a toddler, a child, a young adult, an adult or a geriatric. In some embodiments, a subject to be treated with HSPPC-96 is less than 18 years of age. In some embodiments, a subject to be treated with the combination is 18 years of age or older. In some embodiments, the subject has or is suspected of having a GBM. In some embodiments, a subject to be treated with HSPPC-96 has a confirmed histological diagnosis of GBM. In some embodiments, a subject to be treated with the combination undergoes or has undergone a surgery to remove a GBM tumor.

In some embodiments, a subject to be treated with HSPPC-96 has had ≥90% of a GBM tumor resected prior to the treatment. In some embodiments, a subject to be treated with HSPPC-96 has had ≥80% of a GBM tumor resected prior to the treatment. In some embodiments, a subject to be treated with HSPPC-96 has had ≥70% of a GBM tumor resected prior to the treatment. In some embodiments, a subject to be treated with HSPPC-96 has had ≥60% of a GBM tumor resected prior to the treatment. In some embodiments, a subject to be treated with HSPPC-96 has more than one GBM tumor. In some embodiments, the subject to be treated with HSPPC-96 has had ≥90% of the more than one GBM tumor resected prior to the treatment. In some embodiments, the subject to be treated with HSPPC-96 has had ≥80% of the more than one GBM tumor resected prior to the treatment. In some embodiments, the subject to be treated with HSPPC-96 has had ≥70% of the more than one GBM tumor resected prior to the treatment. In some embodiments, the subject to be treated with HSPPC-96 has had ≥60% of the more than one GBM tumor resected prior to the treatment.

In some embodiments, a subject to be treated has not had radiotherapy within 6 months, within 4 months, within 3 months, within 2 months or within 1 month prior to administration of the combination.

In some embodiments, a subject to be treated has not had a prior treatment with an anti-angiogenic agent targeting the VEGF pathway.

In some embodiments, a subject to be treated has not had a prior treatment with HSPPC-96 or other immunotherapy.

In some embodiments, a subject to be treated with HSPPC-96 has not received a treatment with vincristine, nitrosureas, procarbazine, other chemotherapy, and/or any investigational agent within 16 weeks, 12 weeks, 8 weeks, 6 weeks, 4 weeks, or 2 weeks prior to the treatment with HSPPC-96.

In some embodiments, a subject to be treated with HSPPC-96 has not received a prior adjuvant therapy.

In some embodiments, a subject to be treated with HSPPC-96 has a Karnofsky Performance Status (KPS) greater than 70.

In some embodiments, a subject to be treated with HSPPC-96 has a granulocyte count of ≥1,500/μl. In some embodiments, a subject to be treated with HSPPC-96 has a platelet count of ≥100,000/μl. In some embodiments, a subject to be treated with HSPPC-96 is not lymphopenic. In some embodiments, a subject to be treated with HSPPC-96 has serum creatinine levels of ≤1.5 mg/dl. In some embodiments, a subject to be treated with HSPPC-96 has bilirubin levels that are less than or equal to 1.5 times normal upper limits of clinically normal bilirubin levels, and/or Calculated Creatinine Clearance (CCC) levels that are less than or equal to 2.5 times normal upper limits of clinically normal CCC levels.

In some embodiments, the immune status in peripheral blood samples is assessed to identify subjects who are candidates for treatments comprising HSPPC-96 vaccination. The immune status is evaluated by measuring, e.g., whole blood cell count, absolute lymphocyte count, monocyte count, percentage of CD4⁺CD3⁺ T cells, percentage of CD8⁺CD3⁺ T cells, percentage of CD4⁺CD25⁺FoxP3⁺ regulatory T cells and other phenotyping of PBL surface markers. The cell counts that would indicate subjects are eligible for treatments comprising HSPPC-96 vaccination are whole blood cell count (expressed as ×10⁹/L) of, e.g., 3.5, 4, 4.5, 5, 5.5, 6, 6.5, 7, 7.5, 8, 8.5, 9, 9.5, 10, 10.5 or 11; absolute lymphocyte count (expressed as ×10⁹/L) of, e.g., 0.7, 1.0, 1.3, 1.9, 2.2, 2.5, 2.8, 3.1, 3.4, 3.7, 4.1, 4.4 or 4.8; or monocyte count (expressed as ×10⁹/L) of 0.1, 0.2, 0.3, 0.4, 0.5, 0.6, 0.7 or 0.8.

### Patient Evaluation

Patients treated with the cancer vaccine may be tested for an anti-tumor immune response. In this regard, peripheral blood from patients may be obtained and assayed for markers of anti-tumor immunity. Using standard laboratory procedures, leukocytes may be obtained from the peripheral blood and assayed for frequency of different immune cell phenotypes, HLA subtype, and function of anti-tumor immune cells.

The majority of effector immune cells in the anti-tumor response are CD8+ T cells and thus are HLA class I restricted. Using immunotherapeutic strategies in other tumor types, expansion of CD8+ cells that recognize HLA class I restricted antigens is found in a majority of patients. However, other cell types are involved in the anti-tumor immune response, including, for example, CD4+ T cells, and macrophages and dendritic cells, which may act as antigen-presenting cells in the CNS. Populations of T cells (CD4+, CD8+, and Treg cells), macrophages, and antigen presenting cells may be determined using flow cytometry with the HLA subtype of CD8+ T cells determined by a complement-dependent microcytotoxicity test.

To determine if there is an increase in anti-tumor T cell response, an enzyme linked immunospot assay may be performed to quantify the IFNγ-producing peripheral blood mononuclear cells (PBMC). This technique provides an assay for antigen recognition and immune cell function. In some embodiments, subjects who respond clinically to the vaccine may have an increase in tumor-specific T cells and/or IFNγ-producing PBMCs. In some embodiments, immune cell frequency is evaluated using flow cytometry. In some embodiments, HLA-subtype is evaluated using complement-dependent microcytotoxicity test. In some embodiments, antigen recognition and immune cell function is evaluated using enzyme linked immunospot assays.

In some embodiments, a panel of assays may be performed to characterize the immune response generated to HSPPC-96 given in combination with temozolomide and/or radiotherapy.

In some embodiments, the panel of assays includes one or more of the following tests: whole blood cell count, absolute lymphocyte count, monocyte count, percentage of CD4⁺CD3⁺ T cells, percentage of CD8⁺CD3⁺ T cells, percentage of CD4⁺CD25⁺FoxP3⁺ regulatory T cells and other phenotyping of PBL surface markers, intracellular cytokine staining to detect proinflammatory cytokines at the protein level, qPCR to detect cytokines at the mRNA level and CFSE dilution to assay T cell proliferation.

In evaluating a subject, a number of other tests may be performed to determine the overall health of the subject. For example, blood samples may be collected from subjects and analyzed for hematology, coagulation times and serum biochemistry. Hematology for CBC may include red blood cell count, platelets, hematocrit, hemoglobin, white blood cell (WBC) count, plus WBC differential to be provided with absolute counts for neutrophils, eosinophils, basophils, lymphocytes, and monocytes. Serum biochemistry may include albumin, alkaline phosphatase, aspartate amino transferase, alanine amino transferase, total bilirubin, BUN, glucose, creatinine, potassium and sodium. Protime (PT) and partial thromboplastin time (PTT) may also be tested. One or more of the following tests may also be conducted: anti-thyroid (anti-microsomal or thyroglobulin) antibody tests, assessment for anti-nuclear antibody, and rheumatoid factor. Urinalysis may be performed to evaluated protein, RBC, and WBC levels in urine. Also, a blood draw to determine histocompatibility leukocyte antigen (HLA) status may be performed.

In some embodiments, radiologic tumor evaluations are performed one or more times throughout a treatment to evaluate tumor size and status. For example, tumor evaluation scans may be performed within 30 days prior to surgery, within 48 hours or within 72 hours after surgery (e.g., to evaluate percentage resection), 1 week (maximum 14 days) prior to the first vaccination (e.g., as a baseline evaluation), and approximately every 8 weeks thereafter for a particular duration. MRI or CT imaging may be used. Typically, the same imaging modality used for the baseline assessment is used for each tumor evaluation visit.

### Pharmaceutical Compositions

Treatment of human subjects or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the subject, and with the clinical symptoms of the condition to be treated.

The formulation of a therapeutic agent for the treatment disclosed herein may be by any suitable means that results in a concentration of the therapeutic that, combined with other components, e.g., other therapeutic agents, is effective in ameliorating, reducing, or stabilizing a condition to be treated. The agent may be contained in any appropriate amount in any suitable carrier substance, and is may be present in suitable amount (e.g., 1-95% by weight of the total weight of the composition). The composition may be provided in a dosage form that is suitable for parenteral (e.g., subcutaneously, intradermal, intravenously, intramuscularly, or intraperitoneally) administration route. The pharmaceutical compositions may be formulated according to conventional pharmaceutical practice (see, e.g., Remington: The Science and Practice of Pharmacy (21st edition), ed. Lippincott Williams & Wilkins; Twenty-First edition (May 19, 2005) and Encyclopedia of Pharmaceutical Technology (4^{th} edition), eds. J. Swarbrick, Jul. 1, 2013, CRC Press).

As indicated herein, the pharmaceutical compositions according to the disclosure may be in the form suitable for sterile injection. In some embodiments, to prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. In some embodiments, a formulation may also contain one or more preservatives (e.g., methyl, ethyl or n-propyl p-hydroxybenzoate).

### EXAMPLES

### Overview of Examples

Standard therapy for newly diagnosed glioblastoma (GBM) is surgical resection, followed by concurrent radiotherapy and temozolomide chemotherapy. The addition of an autologous heat shock protein vaccine to standard therapy was evaluated. Tumor-induced immunosuppression, mediated by expression of PD-L1 on tumor cells and circulating monocytes in GBM patients, may impact the efficacy of vaccination. Expression of PD-L1 on peripheral monocytes was evaluated for the first time as a predictor of survival. Adult patients with GBM underwent surgical resection followed by standard chemoradiotherapy. Autologous vaccine (Prophage) was generated from resected tumors and delivered in weekly 25 μg vaccinations after completion of radiotherapy, followed by adjuvant temozolomide with further vaccinations. The primary endpoint was overall survival. Forty-six patients received the vaccine with a median overall survival of 23.8 months (95% Confidence Interval [CI], 19.8-30.2). Median overall survival for patients with high PD-L1 expression on monocytes (above the median, 54.5%) was 18.0 months (95% CI, 10.0-23.3) as compared to 44.7 months (95% CI, incalculable) for patients with low PD-L1 expression (below the median, 54.5%) (hazard ratio 3.3; 95% CI, 1.4-8.6; p=0.007). A multivariate proportional hazards model revealed MGMT methylation, Karnofsky performance status, and PD-L1 expression as the primary independent predictors of survival. Vaccination with autologous tumor-derived heat shock proteins may improve survival for GBM patients when combined with standard therapy, and warrants further study. Systemic immunosuppression mediated by peripheral monocyte expression of PD-L1 is a factor that may significantly impact vaccine efficacy.

### Example 1: Materials and Methods

### Study Patients

Patients 18 years of age or older with newly diagnosed, histopathologically confirmed GBM were eligible for participation in the study. Patients were initially screened for inclusion at the time of radiographic diagnosis and underwent surgical resection with collection of tissue for vaccine generation. Additional eligibility criteria included a post-operative Karnofsky performance status (KPS) of at least 70 (on a scale of 0 to 100, with higher numbers indicating improved performance), an extent of surgical resection in excess of 90% of the contrast enhancing tumor on post-operative MRI within 30 days of surgery, e.g., within 3 days of surgery, or within 28 days of surgery, and sufficient tumor tissue collected to generate a minimum of four 25 μg doses of vaccine. Patients were excluded from the study for known systemic autoimmune diseases, primary or secondary immunodeficiency, concurrent malignancy within the past 5 years, a bleeding diathesis, uncontrolled active infection, or other serious unstable medical condition. Following surgical resection, patients received conformal radiotherapy with concurrent temozolomide chemotherapy according to the standard of care. Patients were excluded from receiving vaccine if repeat MRI at the completion of radiotherapy demonstrated evidence of tumor progression. In some embodiments, disease status and tumor response is used to evaluate progression. In some embodiments, disease status is assessed by one or more of the following: clinical evaluation (neurology examination, physical examination, Karnofsky score), radiographic evaluation, and surgical biopsy. In some embodiments, baseline assessment of existing disease and subsequent assessment of tumor responses are based on the Response Assessment in Neuro-Oncology Working (RANO) Group criteria for progression. In some embodiments, progression is assessed using diagnostic imaging. In some embodiments, progression is identified through diagnostic imaging when there is enhancement outside of a radiation field (e.g., beyond a high-dose region or 80% isodose line). In some embodiments, progression is identified when there is evidence of viable tumor on histopathologic sampling. In some embodiments, progression is detected when solid tumor areas (e.g., areas containing greater than 70% tumor cell nuclei) are detected by histopathologic sampling. In some embodiments, progression is detected when a high, or a progressive increase in, MIB-1 proliferation index is detected by histopathologic sampling compared with prior biopsy. In some embodiments, progression is detected when histologic progression or increased anaplasia is detected in a tumor. However, in some embodiments, following twelve weeks after completion of treatment, e.g., with autologous heat-shock protein peptide complex, clinical deterioration not attributable to concurrent medication or comorbid conditions indicates progression. Patients were required to begin vaccine therapy within 2 to 5 weeks following completion of radiotherapy.

### Study Treatment

Patients were registered for participation before surgical resection. All patients underwent aggressive resection with intra-operative collection of tissue to generate autologous vaccine. Fresh frozen tumor tissue was shipped to the vaccine manufacturing facility (Agenus, Inc., Lexington, Mass.) to generate vaccine after confirmation of the diagnosis. Approximately 7 grams of tissue were used to produce a minimum of four 25 μg vaccine doses. Vaccine quality was confirmed by post-production testing according to good manufacturing practice guidelines. Extent of surgical resection and participation eligibility was determined by the principle investigator at each site based on the post-operative MRI. After surgical resection, patients received radiotherapy (60 Gy administered in 2 Gy fractions 5 days a week for 6 weeks) and oral temozolomide (75 mg per square meter of body surface area daily for a maximum of 49 days). At completion of radiotherapy, patients underwent clinical and radiographic evaluation to demonstrate disease stability. Patients with stable disease received the HSPPC-96 vaccine beginning 2 to 5 weeks post radiotherapy. Vaccine was administered in 25 μg doses through intradermal injection every week for 4 weeks. Maintenance temozolomide treatment began 2 weeks after administration of the 4th vaccine at an initial dose of 150 mg per square meter for 5 consecutive days in a 28-day cycle. The dose was increased to 200 mg per square meter for 5 days in subsequent cycles if no treatment-related adverse events greater than grade 2 were noted. The 5th vaccine dose (if available) was given on the same day as the start of maintenance temozolomide. Subsequent vaccine doses were given beginning 3 weeks after the 5th vaccine and administered on a monthly basis. Vaccinations continued until depletion of the vaccine or tumor progression. Maintenance temozolomide was planned for 6 cycles with the option of extension to a total of 12 cycles if there were no significant adverse events. The extent of temozolomide therapy and subsequent treatment at tumor progression was at the discretion of the patient's primary neuro-oncologist.

### Patient Evaluation

Baseline evaluation including neurologic assessment, complete blood analysis, and tumor imaging with contrast-enhanced MRI was performed pre-operatively and immediately postoperatively, usually within 48 hours. KPS was graded by the treating provider. Repeat assessment by physical examination and imaging was performed at the completion of initial chemoradiotherapy, just prior to the first vaccine administration. Final eligibility to receive vaccine was determined at this time point. After initiation of vaccine therapy, patients were evaluated every 4 weeks by clinical examination, and every 8 weeks by radiographic imaging with contrast-enhanced MRI. Tumor progression was assessed according to the modified RANO criteria (Table 1). Adverse events were assessed and graded according to the National Cancer Institute Common Terminology Criteria for Adverse Event (CTCAE), version 4.0.

TABLE 1
Criteria for determining first progression of GBM: modified from Response Assessment
in Neuro-Oncology (RANO) criteria

| Progressive disease <12 weeks | Progressive disease ≥12 weeks |
|---|---|
| after completion of radiotherapy | after completion of radiotherapy |
| 1. New enhancement outside of the radiation field | 1. New contrast enhancing lesion outside of |
| (beyond the 80% isodose line) | radiation field |
| 2. Histopathologic confirmation of viable tumor | 2. Increase by ≥50% of enhancing lesion within the |
| (regions of >70% viable tumor cells) on biopsy of | radiation field while on stable or increasing dose of |
| increased enhancement in the high-dose radiation | corticosteroids |
| field | 3. Clinical deterioration not attributable to |
| | concurrent medication or comorbid condition |

### Immunologic Evaluation

Patients undergoing surgical resection at sites capable of processing of blood for immunologic assays had 30 milliliters of peripheral blood obtained for laboratory analysis at the time of craniotomy. Peripheral blood leukocytes were extracted from whole blood by centrifugation on a Ficoll gradient. Extracted leukocytes were stained with the following fluorescence-conjugated antibodies to identify immunosuppressive monocytes/macrophages within the total leukocyte population: CD45 FITC, CD11b PE-Cy7, PD-L1 PE (eBioscience, San Diego, Calif.). Cell counts and relative fluorescence was measure by flow cytometry on a FACSCalibur cytometer (BD Biosciences, San Jose, Calif.) and analyzed using FlowJo Software (TreeStar, Ashland, Oreg.).

### Study End Points

The primary end point for the study was duration of overall survival, defined as the time from surgical resection to death of any cause. The secondary endpoint was duration of progression-free survival, defined as the time from resection until either documented disease progression or death. All patients were followed on study protocol with regular clinical and radiographic assessment for evidence of progression for 24 months, then peripherally until death. Investigator assessment of medical records beyond the 24 month period was utilized to define further dates of progression.

### Statistical Analysis

The trial was designed to test the alternative hypothesis that median survival for patients treated with the HSPPC-96 vaccine would be 23 months or greater against the null hypothesis that median survival would be 15 months or less. An exponential distribution with a 1 year accrual period and 2 years of follow-up was assumed. To achieve a power of 80% using a log rank test with a two-sided alpha of 10%, a target accrual of 55 patients was planned. The Kaplan-Meier method was used to estimate the survival distributions of the primary and secondary endpoints. To determine the relative impact of vaccine therapy on progression free and overall survival within molecularly defined subgroups, survival analysis was performed on patient subsets separated by MGMT methylation status and peripheral monocyte PD-L1 expression. Differences between groups were assessed using the log-rank test, and a univariate proportional hazards model was fit to calculate hazard ratios. A multivariate proportional hazards model was developed to incorporate other known predictors of survival and vaccine efficacy, including age, KPS, time to vaccination, and number of vaccine doses administered. Differences between groups were accepted as statistically significant for p values less than 0.05.

### Example 2: Clinical Study Results

### Patients

109 patients were screened for enrollment at 8 centers in the United States. Forty-six patients met all pre and post-operative criteria for enrollment and constitute the intention-to-treat population. The baseline characteristics of the 46 treated patients are shown in Table 2. Patients received a median of 9 vaccine doses (range 3-26). Vaccine administration was discontinued in 28 (61%) patients due to depletion of vaccine, 15 (33%) patients due to disease progression, and 3 (6%) patients due to withdrawal from the study (two patients due to non-compliance and one patient due to death). All patients were followed until death or closure of data analysis. No patients were lost to follow-up.

TABLE 2
Baseline Characteristics of Patients

| Characteristic | Enrolled Patients (N = 46) |
|---|---|
| Age, yr | |
| median | 58 |
| range | 30-75 |
| Gender, no. (%) | |
| male | 29 (63%) |
| female | 17 (37%) |
| KPS, no. (%) | |
| 100 | 7 (15%) |
| 90 | 26 (57%) |
| 80 | 11 (24%) |
| 70 | 2 (4%) |
| MGMT status, no. (%) | |
| methylated | 23 (50%) |
| unmethylated | 19 (41%) |
| data unavailable | 4 (9%) |
| Vaccinations Received, no. | |
| median | 9 |
| range | 3-26 |
| Time from surgery to first vaccine, weeks | |
| median | 13.4 |
| range | 10.0-17.1 |
| Reason for discontinuing vaccine, no. (%) | |
| vaccine depleted | 28 (61%) |
| tumor progression | 15 (33%) |
| patient non-compliance | 3 (6%) |

### Treatment Outcomes

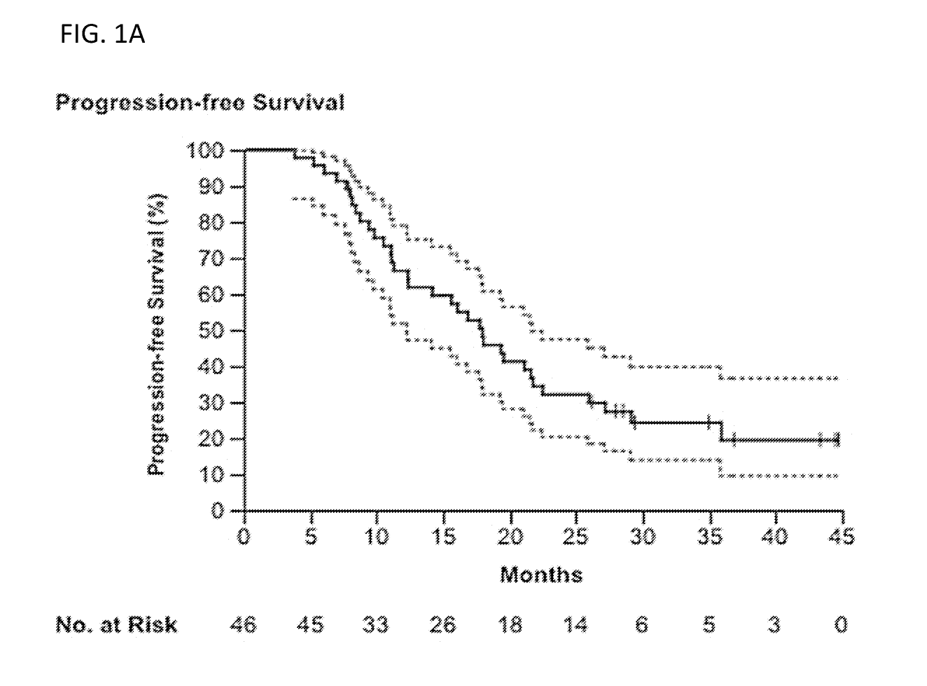

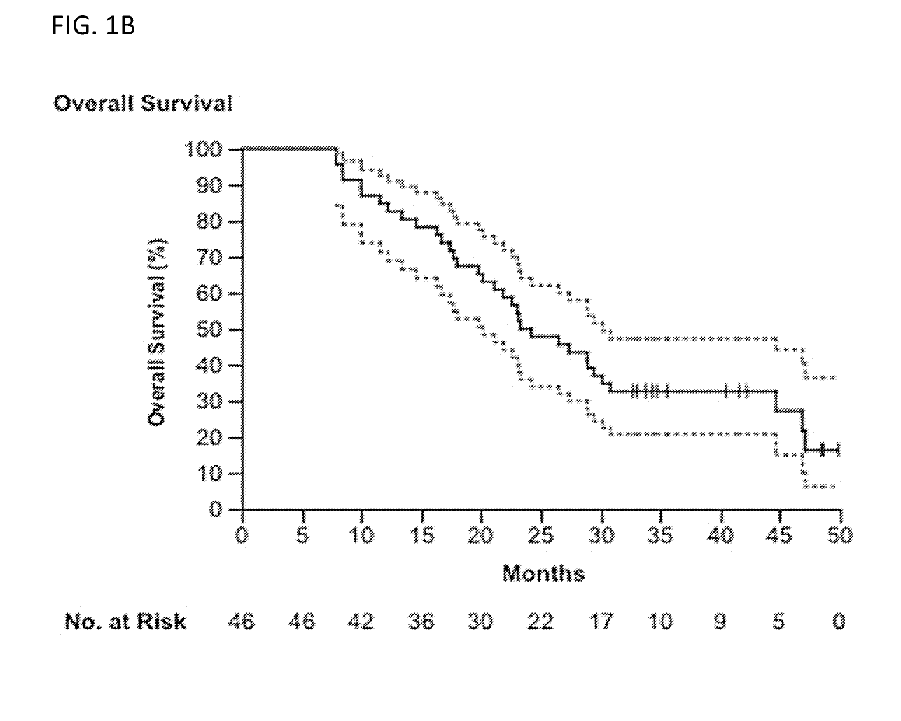

At the time of data analysis, 12 of 46 patients (26%) were alive and 12 of 46 (26%) had no evidence of progression. Median progression-free survival was 18.0 months (95% Confidence Interval [CI], 12.4-21.8) (FIG. 1A). Median overall survival was 23.8 months (95% CI, 19.8-30.2) with actuarial 2-year survival of 50.0% (95% CI, 35.1% to 64.9%) and 3-year survival of 32.6% (95% CI, 20.0% to 48.1%) (FIG. 1B).

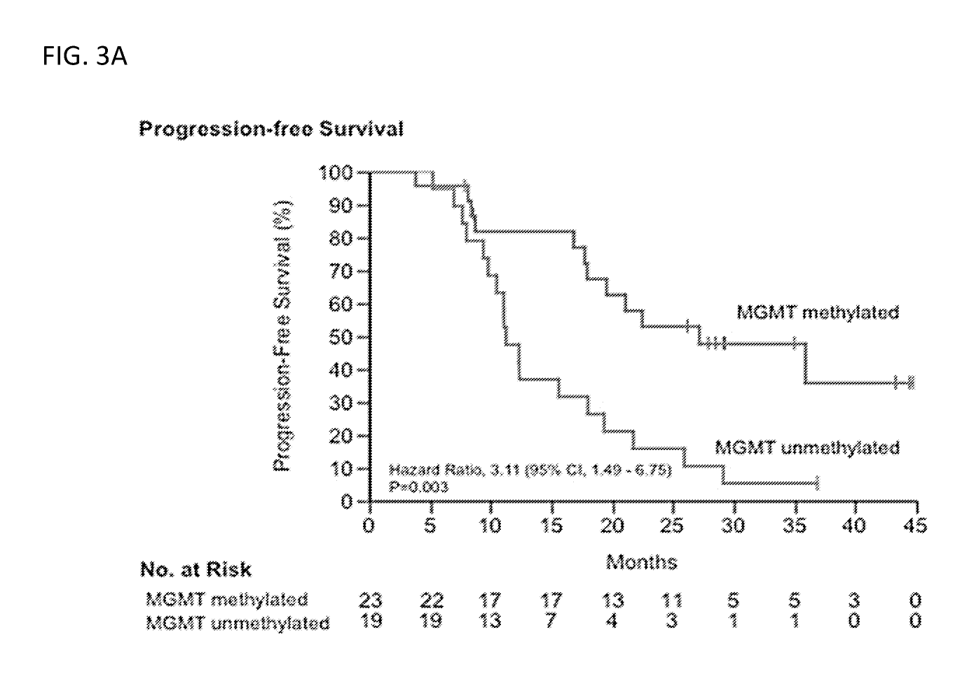

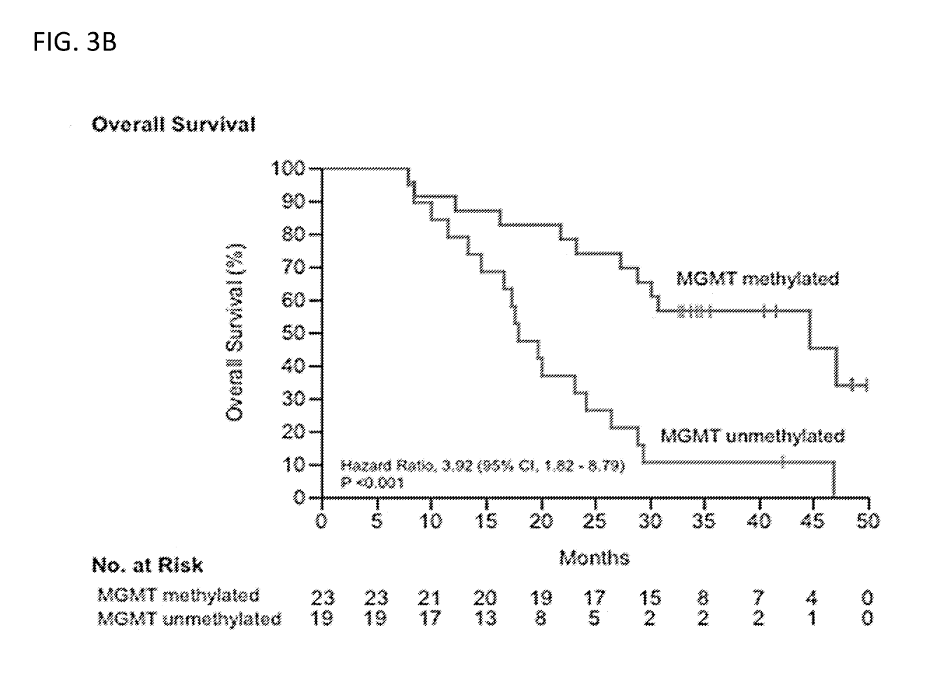

MGMT methylation status was evaluated as a prognostic factor for overall survival. The MGMT promoter was found to be methylated in 23 of 46 patients (50%), with methylation status unavailable for 4 patients (9%). The median overall survival for MGMT unmethylated tumors was 18.0 months (95% CI, 13.4-24.2) as compared to 44.7 months (95% CI, 27.4—incalculable) for MGMT methylated tumors (hazard ratio for death in unmethylated tumors 3.9; 95% CI, 1.8-8.8; p<0.001) (FIGS. 3A-3B).

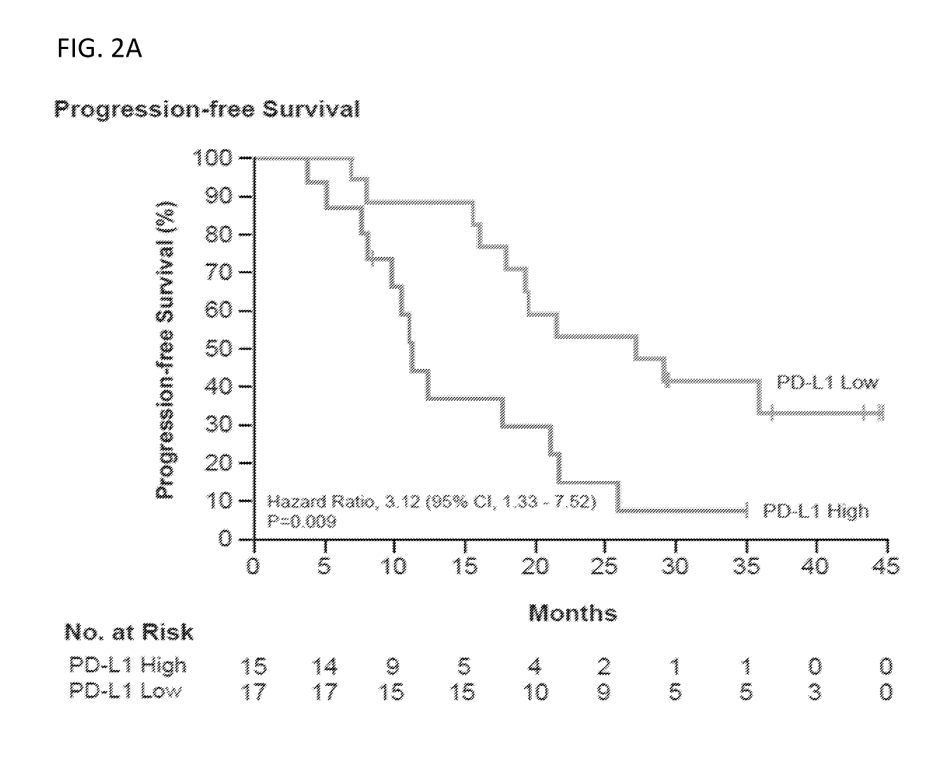

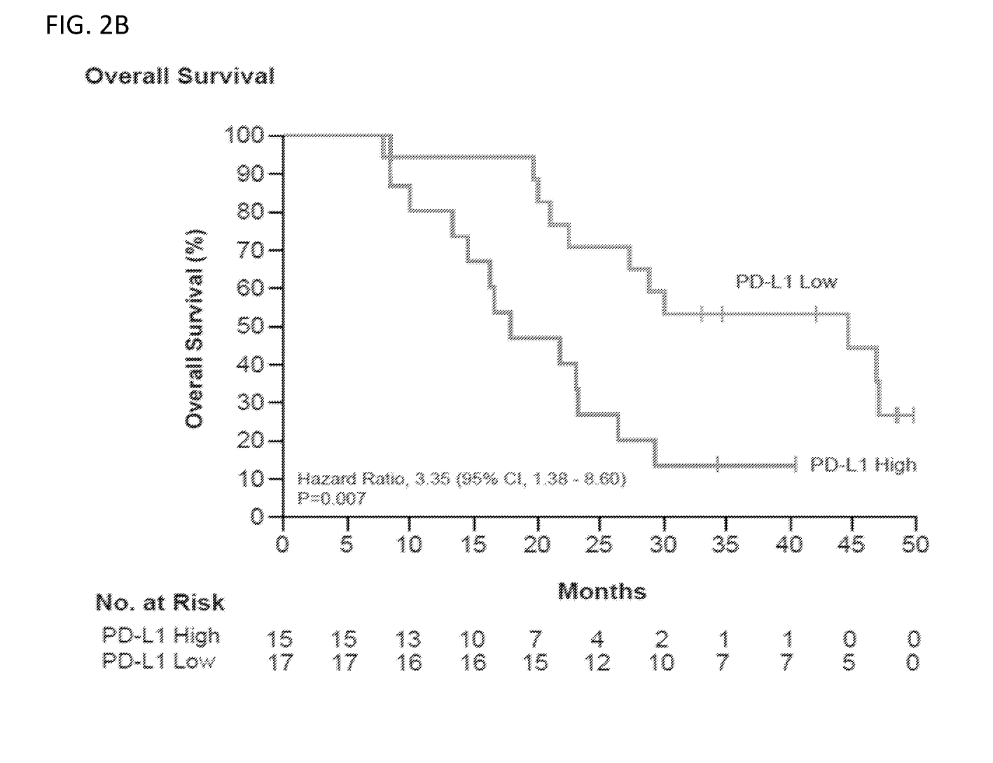

Expression of PD-L1 on circulating peripheral monocytes may be a contributor to tumor-induced immunosuppression. Therefore, PD-L1 expression on peripheral monocytes was evaluated as a prognostic factor for overall survival. Circulating peripheral monocytes (CD45+/CD11b+) obtained from patients at the time of surgery were analyzed for PD-L1 expression to determine the percent of monocytes positive for PD-L1 (cutoff for positivity determined relative to isotype control). Peripheral blood was available for analysis from 32 vaccine-treated patients. Median PD-L1 positivity in circulating monocytes was 54.5% (range 5.9% to 91.8%). Patients were categorized as high PD-L1 expressors (% PD-L1 greater than the median) or low PD-L1 expressors (% PD-L1 less than or equal to the median). The median progression-free survival for high PD-L1 expressors was 11.3 months (95% CI, 7.7-21.1) as compared to 27.2 months (95% CI, 16.1—incalculable) for low PD-L1 expressors (hazard ratio for progression in high expressors 3.1; 95% CI, 1.3-7.5; p=0.009) (FIG. 2A). The median overall survival for high PD-L1 expressors was 18.0 months (95% CI, 10.0-23.3) as compared to 44.7 months (95% CI, 21.1—incalculable) for low PD-L1 expressors (hazard ratio for death in high expressors 3.3; 95% CI, 1.4-8.6; p=0.007) (FIG. 2B).

A multivariate proportional hazards model was developed for overall survival incorporating MGMT methylation status, PD-L1 expression in monocytes, and previously reported predictors of outcome (age, KPS) as well as vaccine administration factors (number of vaccines administered, time to first vaccination) (Table 3). Only MGMT methylation (hazard ratio for unmethylated tumors 6.3; 95% CI, 2.1-22.0; p<0.001), PD-L1 expression (hazard ratio for high expression 4.0; 95% CI, 1.4 to 12.7; p=0.008), and KPS (hazard ratio for KPS<90 5.1; 95% CI, 1.5-18.0; p=0.009) were found to be independent predictors of overall survival.

TABLE 3
Proportional Hazards Model of Overall Survival

| Variable | Hazard Ratio (95% CI) | P value |
|---|---|---|
| Age (per 1-yr increment) | 1.06 (0.99-1.13) | 0.08 |
| MGMT | | |
| methylated | reference | |
| unmethylated | 6.3 (2.1-22.0) | <0.001 |
| PD-L1 expression | | |
| Low expression | reference | |
| High expression | 4.0 (1.4-12.7) | 0.008 |
| KPS | | |
| 90-100 | reference | |
| 70-80 | 5.1 (1.5-18.0) | 0.009 |
| Time to 1^{st} vaccine | 1.4 (0.9-2.2) | 0.13 |
| (per week increment) | | |
| Number of vaccines given | 0.97 (0.86-1.08) | 0.61 |
| (per vaccine increment) | | |

### Adverse Events

All patients were followed for 24 months for safety. Adverse events of any type and severity were identified in 44 of 46 patients (96%) (Table 4). Adverse events attributable to the vaccine were identified in 34 patients (74%) with no serious (grade 3 or 4) events related to vaccination. The most common vaccine related events were minor injection site reactions or constitutional symptoms.

TABLE 4
Adverse Events

| | Any Adverse Event | Grade 3-4 Event |
|---|---|---|
| Adverse Events | no. of patients (%) | no. of patients (%) |
| Any event | 44 (95.7%) | 31 (67.4%) |
| TWZ-related event | 42 (91.3%) | 21 (45.6%) |
| Vaccine-related event | 34 (73.9%) | 0 (0%) |
| Vaccine-related events | | |
| Constitutional | | |
| Fatigue | 4 (8.7%) | 0 (0%) |
| Flu-like illness | 2 (4.3%) | 0 (0%) |
| Injection Site Reaction | | |
| Induration | 1 (2.2%) | 0 (0%) |
| Pruritus | 1 (2.2%) | 0 (0%) |
| Erythema | 16 (34.8%) | 0 (0%) |
| Other reaction | 8 (17.4%) | 0 (0%) |
| Gastrointestinal | | |
| Diarrhea | 1 (2.2%) | 0 (0%) |
| Metabolic | | |
| Anorexia | 1 (2.2%) | 0 (0%) |
| Musculoskeletal | | |
| Myalgia | 1 (2.2%) | 0 (%) |
| Nervous | | |
| Dizziness | 1 (2.2%) | 0 (0%) |
| Headache | 1 (2.2%) | 0 (0%) |
| Skin | | |
| Erythema | 3 (6.5%) | 0 (0%) |
| Pruritus | 2 (4.3%) | 0 (0%) |
| Rash | 1 (2.2%) | 0 (0%) |
| Vascular | | |
| Flushing | 1 (2.2%) | 0 (0%) |

### MGMT/PD-L1 Analysis

Table 5 shows Median OS calculated using the Kaplan Meier estimate. Tables 6-9 show quartile estimates from The SAS System®. These data were computed using the LIFETEST procedure of The SAS System®, which can be used to compute nonparametric estimates of the survivor function by the product-limit method (also called the Kaplan-Meier method).

TABLE 5
Median, Range, N, and Outputs

| | Methylated | Un Methylated | Total (n = 42) |
|---|---|---|---|
| PD-L1 high | 22.6, | 16.7 | 18.0 |
| | Range: 8.5-40.5 | Range: 8.4-29.4 | Range: 8.4-40.5 |
| | n = 6, alive = 2 | n = 9, alive = 0 | N = 15, alive = 2 |
| PD-L1 low | 47.1 | 24.6 | 44.7 |
| | Range: 27.4-52.5 | Range: 7.9-46.9 | Range: 7.9-52.5 |
| | n = 9, alive = 5 | N = 6, alive = 1 | N = 17, alive = 6 |
| Total | 44.7 | 18.0 | 23.8 |
| (n = 32) | Range: 7.9-52.5 | Range: 7.9-46.9 | Range: 7.9-52.5 |
| | N = 23, alive = 11 | N = 19, alive = 1 | n = 46 alive = 12 |

TABLE 6
The SAS System
The LIFETEST Procedure
Stratum 1: group = High PD-L1/methylated
Quartile Estimates

| | Point | 95% Confidence Interval | | |
|---|---|---|---|---|
| Percent | Estimate | Transform | [Lower | Upper) |
| 75 | | LOGLOG | 16.3333 | |
| 50 | 22.5833 | LOGLOG | 8.5000 | |
| 25 | 16.3333 | LOGLOG | 8.5000 | 23.3000 |

TABLE 7
The SAS System
The LIFETEST Procedure
Stratum 2: group = High PD-L1/unmethylated
Quartile Estimates

| | Point | 95% Confidence Interval | | |
|---|---|---|---|---|
| Percent | Estimate | Transform | [Lower | Upper) |
| 75 | 23.1667 | LOGLOG | 14.6000 | 29.4333 |
| 50 | 16.7000 | LOGLOG | 8.4333 | 26.5000 |
| 25 | 13.4000 | LOGLOG | 8.4333 | 16.7000 |

TABLE 8
The SAS System
The LIFETEST Procedure
Stratum 3: group = Low PD-L1/methylated
Quartile Estimates

| | Point | 95% Confidence Interval | | |
|---|---|---|---|---|
| Percent | Estimate | Transform | [Lower | Upper) |
| 75 | | LOGLOG | 44.6667 | |
| 50 | 47.1000 | LOGLOG | 27.3667 | |
| 25 | 44.6667 | LOGLOG | 27.3667 | |

TABLE 9
The SAS System
The LIFETEST Procedure
Stratum 4: group = Low PD-L1/unmethylated
Quartile Estimates

| | Point | 95% Confidence Interval | | |
|---|---|---|---|---|
| Percent | Estimate | Transform | [Lower | Upper) |
| 75 | 46.8667 | LOGLOG | 19.8000 | 46.8667 |
| 50 | 24.5500 | LOGLOG | 7.9000 | 46.8667 |
| 25 | 19.8000 | LOGLOG | 7.9000 | 28.9333 |

Summary of the Number of Censored and Uncensored Values

| | | | | | Percent |
|---|---|---|---|---|---|
| Stratum | group | Total | Falled | Censored | Censored |
| 1 | High | 6 | 4 | 2 | 33.33 |
| | PD-L1/methylated | | | | |
| 2 | High | 9 | 9 | 0 | 0.00 |
| | PD-L1/unmethylated | | | | |
| 3 | Low | 9 | 4 | 5 | 55.56 |
| | PD-L1/methylated | | | | |
| 4 | Low | 8 | 5 | 1 | 16.67 |
| | PD-L1/unmethylated | | | | |
| Total | | 30 | 22 | 8 | 26.67 |

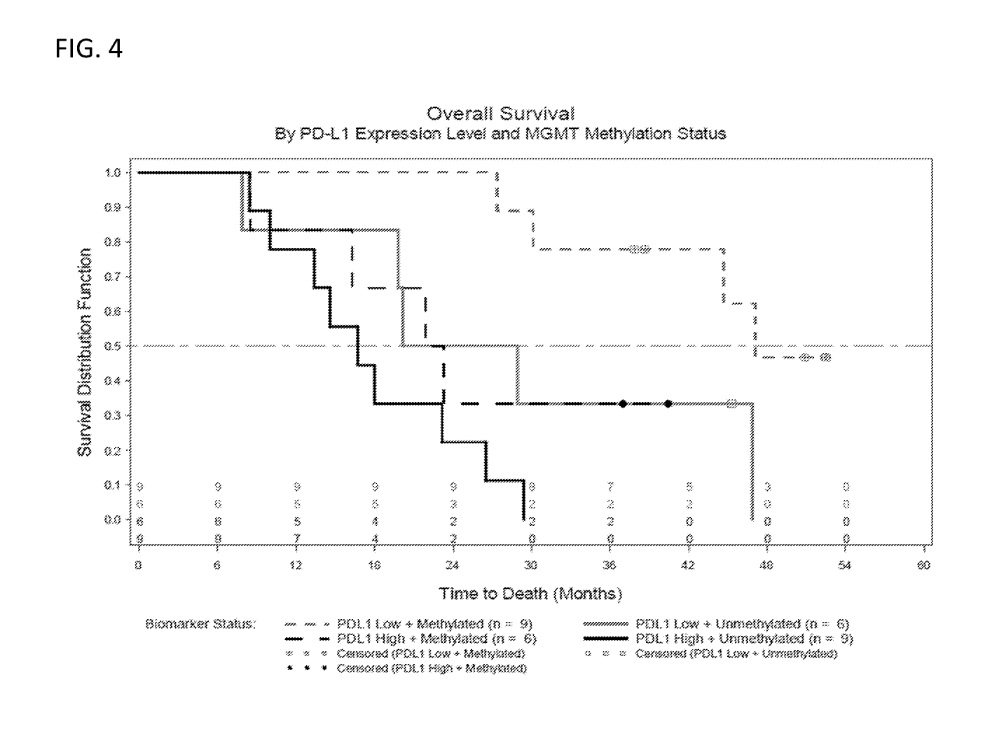

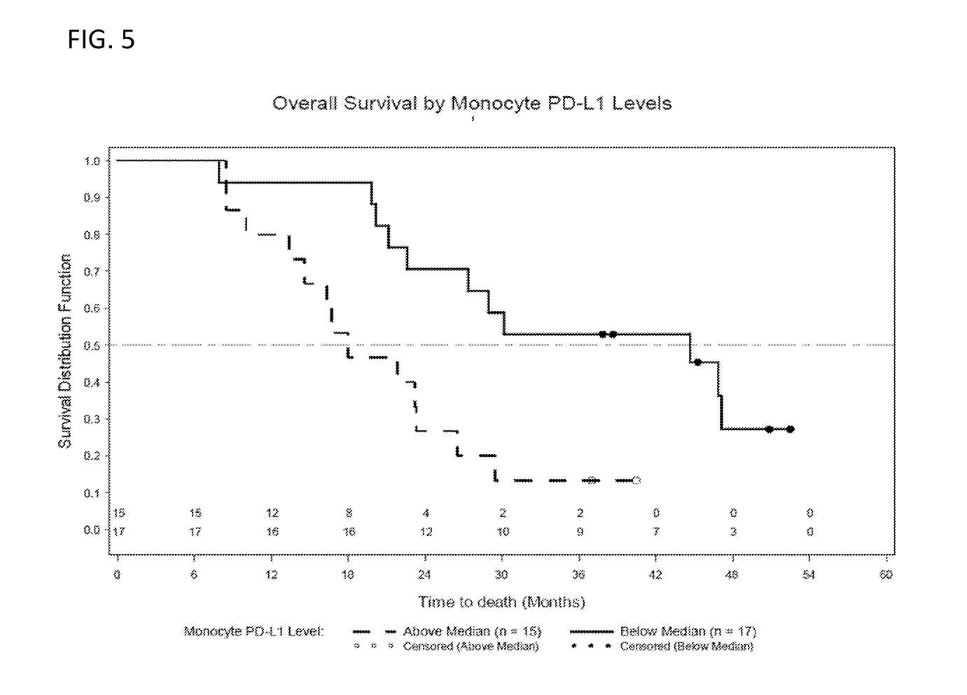

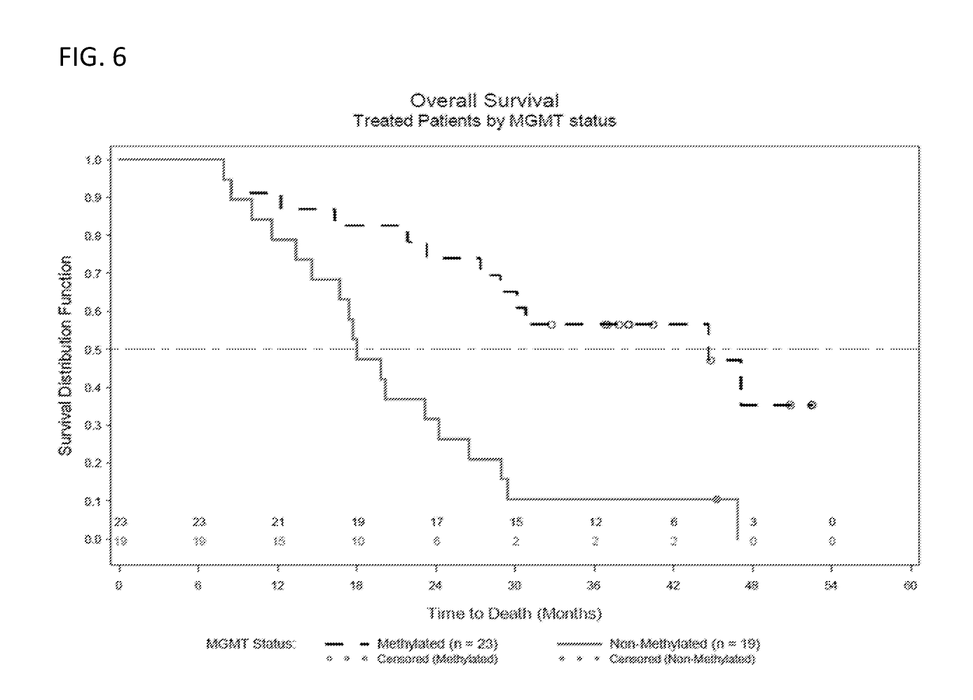

Tables 10-12 show summaries of survival data in treated patients. FIGS. 4-6 correspond, in order, to the data listed below.

TABLE 10
Overall Survival
Treated Patients

| | HSPPC-96 |
|---|---|
| | (N = 46) |
| Number (%) of Patients: | 46 (100.0%) |
| With an Event (Death) | 34 (73.9%) |
| Censored | 12 (26.1%) |
| Median Follow-up Time (Months) | 23.75 |
| Min.-Max. | (7.90, 52.53) |
| Overall Survival Time (Months) @ | |
| 25th Percentile | 16.70 |
| Median | 23.75 |
| 75th Percentile | 46.87 |

TABLE 11
Overall Survival by Monocyte PD-L1 Levels

| | Above Median | Below Median | | Hazard Ratio |
|---|---|---|---|---|
| | Group | Group | | Above Median:Below Median |
| | (N = 15) | (N = 17) | P-Value # | (65% CI) $ |
| Number (%) of Patients: | 15 (100.0%) | 17 (100.0%) | | |
| With an Event (death) | 13 (86.7%) | 11 (64.7%) | | |
| Censored | 2 (13.3%) | 6 (35.3%) | | |
| Median Follow-up Time (Months) | 18.00 | 37.87 | | |
| Min.-Max. | (8.43, 40.47) | (7.90, 52.53) | | |
| Overall Survival Time (Months) @ | | | | |
| 25th Percentile | 13.40 | 22.60 | 0.003 | 3.347 |
| Median | 18.00 | 44.67 | | (1.361, 8.227) |
| 75th Percentile | 26.50 | — | | |

TABLE 12
Overall Survival
Treated Patients by MGMT status

| | Methylated | Non-Methylated | | Hazard Ratio |
|---|---|---|---|---|
| | Group | Group | | Methylated:Non-Methylated |
| | (N = 23) | (N = 19) | P-Value # | (95% CI) $ |
| Number (%) of Patients: | 23 (100.0%) | 19 (100.0%) | | |
| With an Event (Death) | 12 (52.2%) | 18 (94.7%) | | |
| Censored | 11 (47.8%) | 1 (5.3%) | | |
| Median Follow-up Time (Months) | 36.83 | 18.00 | | |
| Min.-Max. | (7.90, 52.53) | (7.90, 46.87) | | |
| Overall Survival Time (Months) @ | | | | |
| 25th Percentile | 23.30 | 13.40 | 0.000 | 0.260 |
| Median | 44.67 | 18.00 | | (0.120, 3.566) |
| 75th Percentile | — | 26.50 | | |

### Discussion of Results

Vaccination with autologous tumor-derived heat shock protein peptide complexes stimulates a specific anti-tumor response in treated patients. These results prompted a phase 2 trial of the vaccine in recurrent GBM with modest improvement in survival over historical controls. The current study was designed to evaluate the benefit of autologous HSPPC-96 vaccination in combination with standard radiation and chemotherapy for patients with newly diagnosed GBM. Overall survival and progression free survival were selected as a primary endpoint.

The current standard of care for newly diagnosed GBM was established by Stupp and colleagues in a phase 3 trial of radiation and temozolomide in which they reported a median overall survival of 14.6 months. Recently, two randomized phase 3 clinical trials of bevacizumab combined with standard chemoradiotherapy for newly diagnosed GBM (RTOG 0825 and AVAGlio) have reported no significant benefit of the addition of bevacizumab. Median overall survival in the placebo groups from both studies ranged from 16.1 to 16.7 months. Compared to these results, patients treated with the HSPPC-96 vaccine had a median overall survival of 23.3 months with nearly 33% of patients surviving greater than 3 years. It is noted that patients in the vaccine study had a better pre-treatment prognosis as a result of a more complete surgical resection. The extent of surgical resection is a positive predictor of outcome for GBM patients, and all patients in the current study had at least 90% extent of resection. However, in the RTOG 0825 study, greater than 60% of patients had a complete resection and the remainder had at least a partial resection, with biopsy-only patients excluded from the analysis.

MGMT methylation was found in 50% of vaccine treated patients compared to 30% in the bevacizumab trials. To ensure that MGMT methylation status alone did not account for the better than expected survival in vaccine-treated patients, overall survival was analyzed by MGMT methylation status. It was found that patients having MGMT promoter methylation had significantly improved survival compared to unmethylated patients, but both groups had better than expected survival compared to historical data. In the bevacizumab trials, median overall survival was 25.0 months in methylated patients and 14.6 months in unmethylated patients. In the current study, median overall survival was surprisingly 44.7 months for methylated patients and 18.0 for unmethylated patients, indicating that vaccinated patients have received clinical benefit beyond what was expected from chemotherapy alone, based on molecular phenotype.

The efficacy of an immunotherapeutic approach relates to an ability to induce a systemic antitumor response. In some cases, GBM is highly immunosuppressive, inhibiting peripheral T cell activation and cytotoxic function. The mechanisms of glioma-induced immunosuppression are multifactorial, but may involve modulation of key immune checkpoints now recognized as important regulators of immunity in many cancers. PD-L1 is a surface protein expressed by leukocytes that binds to the programmed death 1 (PD-1) receptor on activated T cells, inducing T cell anergy and/or apoptosis. It has been recognized that most solid organ cancers, including GBM, can express PD-L1 on the tumor cell surface, inducing local T cell immunoresistance within the tumor microenvironment. Inhibitors targeting PD-L1 and PD-1 have shown clinical efficacy in multiple cancers and are now approved for use in melanoma and lung cancer. The majority of literature on the role of PD-L1 in tumor-induced immunosuppression has focused on tumor expression of the protein, which acts in the tumor microenvironment, but should not impact peripheral immunity.

It has been found that circulating monocytes in most GBM patients have upregulated PD-L1 expression, mediated by a tumor-derived cytokine. Although similar findings have been documented in a small number of studies from other cancers, direct evidence of the immunologic impact of this expression on anti-tumor immunity has not been demonstrated. In this study, patients with the most elevated expression of PD-L1 on circulating monocytes were found to have more systemic immunosuppression and less response to vaccination. A decrease in median overall survival of over 24 months was found for patients with high peripheral PD-L1 expression compared to those with low peripheral PD-L1 expression. The impact of PD-L1 expression was independent of MGMT methylation status, and was found to be highly predictive of outcome in multivariate analysis. This novel finding indicates that PD-L1 expression in monocytes may be as or more important than tumor surface expression. This finding also indicates that PD-L1 status in monocytes is useful as a stratification factor in immunotherapy trials for GBM. Additionally, combining a PD-L1 inhibitor (e.g., an anti-PD-L1 antagonist antibody) and/or PD-1 inhibitor (e.g., an anti-PD-1 antagonist antibody) with vaccination may improve overall survival, particularly in high PD-L1 expressors. Accordingly, in some embodiments, a PD-L1 inhibitor (e.g., an anti-PD-L1 antagonist antibody) and/or PD-1 inhibitor (e.g., an anti-PD-1 antagonist antibody) may be administered in conjunction with vaccination in high PD-L1 expressors. However, in other embodiments, a PD-L1 inhibitor (e.g., an anti-PD-L1 antagonist antibody) and/or PD-1 inhibitor (e.g., an anti-PD-1 antagonist antibody) may be administered in conjunction with vaccination in low PD-L1 expressors.

Thus, vaccination with an autologous heat shock protein peptide complex vaccine in combination with standard therapy may improve survival for patients with GBM. Expression of PD-L1 on circulating monocytes in GBM patients impacts systemic immunity and efficacy of vaccination.

### Example 3: Study Comparisons

Table 13 provides a comparison of the results of the study disclosed in the Examples herein compared with different GBM treatments. These results show that HSPPC-96 plus Radiotherapy (Rad) and temozolomide treatment (TMZ) in patients having had a GBM tumor surgically removed, as described herein, results in 1) a median overall survival of approximately 23.8 months, which is significantly greater than the a median overall survival of approximately 14.6 months achieved with the standard of care alone; 2) a median overall survival for patients with low PD-L1 expression on peripheral blood monocytes of approximately 44.7 months, which is significantly greater than the median overall survival of approximately 18.0 months observed in high PD-L1 expressors; and 3) a post progression survival of approximately 17.5 months for patients with low PD-L1 expression on peripheral blood monocytes, which is significantly greater than the post progression survival of approximately 6 months for all subjects regardless of PD-L1 expression status or an estimated post-progression survival of approximately 6-12 months observed across different treatments.

TABLE 13

| | | | Estimated Post- |
|---|---|---|---|
| | Median | Median | Progression |
| | PFS | Survival | Survival |
| Data Set | (months) | (months) | (months) |
| HSPPC-96 plus Rad/TMZ as | 18.0 | 23.8 | 5.8 |
| described in Examples | | | |
| herein. | | | |
| HSPPC-96 plus Rad/TMZ as | 27.2 | 44.7 | 17.5 |
| described in Examples | | | |
| herein - low PD-L1 | | | |
| expression | | | |
| Rad/TMZ | 6.9 | 14.6; | 7.7 |
| (Stupp et al., NEJM 2005, | | 18.8 in GTR | |
| LancetOnc 2009) | | | |
| Rad/TMZ ® (Placebo; | 6.2, 10.6 | 16.7, 16.8 | 10.5, 6.2 |
| Avastin) | | | |
| (AVAglio, Chinot et al, | | | |
| NEJM 2014) | | | |
| Rad/TMZ (placebo, Avastin) | 7.3, 10.7 | 15.7, 16.1 | 8.4, 5.4 |
| (RTOG 0825 Gilbert, et al. | | | |
| NEJM 2014) | | | |
| Gliadel ® | 5.9 | 13.9 | 8 |
| (Westphal et al., Neuro- | | | |
| oncology, 2002) | | | |
| ICT-107 control arm in a | 9.0 | 16.7 | 7.7 |
| randomized trial (ASCO | | (73% GTR) | |
| 2014) | | | |
| CDX-110 (P2, n = 18 | 14 | 26 | 12 |
| EGFRVIII) | | | |
| (Sampson et al., JCO 2010) | | | |

Table 14 provides a comparison of the results of the study disclosed in the Examples herein with different GB M treatments based on MGMT promoter methylation status.

TABLE 14

| | Median Survival (months) | |
|---|---|---|
| Data Set | Unmethylated | Methylated |
| Prophage (N = 46) | 18.0 | 44.7 |
| Prophage - Low PD-L1 (N = 17) | 24.6 | 47.1 |
| Prophage - High PD-L1 (N = 15) | 16.7 | 22.6 |
| Radiation/Temodar ® (N = 287) | 12.6 | 23.4 |
| (Stupp et al., NEJM 2005 & | | |
| LancetOnc 2009) | | |
| Radiation/Temodar ®/ | 14.3 | 23.2 |
| Avastin ® (RTOG 0825 | | |
| Trial, Gilbert, NEJM 2013) (N = 637) | | |
| Radiation/Temodar ® in MGMT | 13.4 | 26.3 |
| unmethylated only (control arms of | | |
| Cilengitide trials (N = 89 & 273) | | |
| Nabors et al., NeuroOnc 2015; | | |
| Stupp et al., Lancet Onc 2014) | | |
| Radiation/Temodar ®/ | 17.9 | 29.3 |
| Rindopepimut (N = 65) | | |
| (ACTIII - Schuster et al., NeuroOnc 2015) | | |

### Example 4: Data from a Further Follow-Up

Patients described in Example 2 were followed up after the initial data analysis. Table 15 shows median overall survival calculated using the Kaplan Meier estimate based on updated data.

TABLE 15
Median, Range, N, and Outputs

| | Methylated | Un Methylated | Total (n = 42) |
|---|---|---|---|
| PD-L1 high | 22.6, | 16.7 | 18.0 |
| | Range: 8.5-54.9 | Range: 8.4-29.4 | Range: 8.4-54.9 |
| | N = 6, alive = 2 | N = 9, alive = 0 | N = 15, alive = 2 |
| PD-L1 low | 47.1 | 24.6 | 44.7 |
| | Range: 27.4-61.4 | Range: 7.9-51.3 | Range: 7.9-61.4 |
| | N = 9, alive = 4 | N = 6, alive = 1 | N = 17, alive = 5 |
| Total | 44.7 | 18.0 | 23.8 |
| (n = 32) | Range: 7.9-61.4 | Range: 7.9-51.3 | Range: 7.9-61.4 |
| | N = 23, alive = 9 | N = 19, alive = 1 | N = 46, alive = 10 |

Having thus described several aspects of at least one embodiment of this disclosure, it is to be appreciated that various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only and the disclosure is described in detail by the embodiments that follow.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

Use of ordinal terms such as “first,” “second,” “third,” etc., in the embodiments to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

The entire contents of all references, publications, abstracts, and database entries cited in this specification are incorporated by reference herein.

1. A method for treating a subject who has had a Glioblastoma Multiforme (GBM) tumor surgically removed, the method comprising:
administering to the subject an autologous heat-shock protein peptide complex that comprises peptides derived from the GBM tumor complexed with heat-shock proteins, wherein:
(i) the subject exhibits low PD-L1 expression on peripheral monocytes derived from a sample of the subject's blood; and
(ii) the subject survives at least 36 months following surgical removal of the GBM tumor.

2. The method of claim 1, wherein the subject survives at least 44.7 months following surgical removal of the GBM tumor.

3. The method of claim 1, wherein 60% or less, 54.5% or less, or 50% or less of the peripheral monocytes are PD-L1 positive.

4. The method of claim 1, wherein the peripheral monocytes are selected from the group consisting of CD45+ monocytes, CD11b+ monocytes, and CD45+/CD11b+ monocytes.

5-16. (canceled)

17. A method for treating a subject who has had a Glioblastoma Multiforme (GBM) tumor surgically removed, the method comprising:
(a) selecting a subject as a candidate for a treatment that comprises administration of an autologous heat-shock protein peptide complex that comprises GBM tumor peptides complexed with heat-shock proteins, wherein the selection is based on a determination that the subject is a member of a population having a median post-progression survival of at least 12 months in response to the treatment; and
(b) based on the selection in (a), administering to the subject the autologous heat-shock protein peptide complex.

18. A method for treating a subject having a Glioblastoma Multiforme (GBM) tumor, the method comprising:
administering to the subject an autologous heat-shock protein peptide complex that comprises peptides derived from the GBM tumor complexed with heat-shock proteins, wherein:
a) a sample of the subject's blood was obtained, prior to the administering step, wherein it was determined from the blood sample that less than a threshold level of circulating CD45+/CD11b+ monocytes in the blood of the subject were PD-L1 positive, and wherein the subject was selected as a candidate for administration of the autologous heat-shock protein peptide complex based on that determination, indicating that the subject is a member of a population having a median overall survival of at least 36 months following surgical removal of the GBM tumor in response to the treatment; or
b) the subject survives at least 36 months following surgical removal of the GBM tumor, wherein the subject is selected for the treatment based on detection of low PD-L1 expression on peripheral leukocytes derived from a sample of the subject's blood; or
c) prior to the administering step, it was determined that GBM tumor was MGMT promoter methylation positive, and wherein the subject was selected as a candidate for administration of the autologous heat-shock protein peptide complex based on that determination, indicating that the subject is a member of a population having a median overall survival of at least 44.7 months following surgical removal of the GBM tumor in response to the treatment; or
d) prior to the administering step, it was determined that GBM tumor was MGMT promoter methylation negative, and wherein the subject was selected as a candidate for administration of the autologous heat-shock protein peptide complex based on that determination, indicating that the subject is a member of a population having a median overall survival of at least 18 months following surgical removal of the GBM tumor in response to the treatment; or
e) the subject survives at least 36 months following surgical removal of the GBM tumor, wherein the subject is selected for the treatment based on detection of i) low PD-L1 expression on peripheral leukocytes derived from a sample of the subject's blood, and ii) high MGMT promoter methylation in cells of the GBM tumor; or
f) it was determined from a sample of the subject's blood that greater than a threshold level of circulating CD45+/CD11b+ cells in the blood of the subject were PD-L1 positive; and the method further comprises administering to the subject an effective amount of a PD-1 inhibitor or PD-L1 inhibitor.

19-24. (canceled)

25. The method of claim 18, wherein the subject is selected for the treatment based on detection of i) low PD-L1 expression on peripheral leukocytes derived from a sample of the subject's blood, and ii) high MGMT promoter methylation in cells of the GBM tumor, and the subject survives at least 44.7 months following surgical removal of the GBM tumor.

26. The method of claim 25, wherein 60% or less, 54.5% or less, or 50% or less of the peripheral leukocytes are PD-L1 positive.

27. The method of claim 25, wherein the peripheral leukocytes are selected from the group consisting of CD45+ leukocytes, CD11b+ leukocytes, and CD45+/CD11b+ leukocytes.

28-36. (canceled)

37. The method of claim 1, wherein the sample of the subject's blood is taken within 10 days of the surgical removal of the GBM tumor, wherein optionally the sample of the subject's blood is taken within 24 hours of the surgical removal of the GBM tumor.

38-43. (canceled)

44. The method of claim 1, wherein the subject is administered radiotherapy directed at the area from which the GBM tumor was resected.

45. The method of claim 44, wherein the radiotherapy is completed within 5 weeks of the autologous heat-shock protein peptide complex administration.

46-48. (canceled)

49. The method of claim 44, wherein the subject is further administered oral temozolomide to treat the GBM tumor.

50. The method of claim 49, wherein the oral temozolomide is administered at a dose of 75 mg per square meter of body-surface area during radiotherapy.

51. The method of claim 50, wherein oral temozolomide doses are administered to the subject daily for up to 49 days.

52-60. (canceled)

61. The method of claim 1, wherein the extent of surgical resection of the GBM tumor is in excess of 90%, wherein optionally the extent of surgical resection is as determined by detection of residual contrast-enhancing tumor on post-operative MRI within 30 days of surgery.

62-64. (canceled)

65. The method of claim 1, further comprising administering to the subject the autologous heat-shock protein peptide complex once a week for the first 4 weeks of administration.

66-67. (canceled)

68. The method of claim 1, wherein the subject did not have a concurrent malignancy within the past 5 years of the treatment.

69. (canceled)

70. The method of claim 1, wherein the autologous heat-shock protein peptide complex comprises gp96.

71. The method of claim 1, wherein the autologous heat-shock protein peptide complex is administered by intradermal injection.

72-112. (canceled)
